(19) United States
(12) Patent Application Publication
(10) Pub. No.: US 2015/0104455 A1
(43) Pub. Date: Apr. 16, 2015

(54) METHODS AND COMPOSITIONS FOR TREATING AND DIAGNOSING ACUTE MYOCARDIAL INFARCTION

(71) Applicant: GENERA ISTRAZIVANJA d.o.o., Kalinovica (HR)

(72) Inventors: Slobodan Vukicevic, Zagreb (HR); Lovorka Grgurevic, Zagreb (HR); Ivo Dumic-Cule, Zagreb (HR)

(21) Appl. No.: 14/396,020

(22) PCT Filed: Apr. 25, 2013
(86) PCT No.: PCT/US2013/038294

§ 371 Date: Oct. 21, 2014

Related U.S. Application Data
(60) Provisional application No. 61/638,373, filed on Apr. 25, 2012.
(60) Provisional application No. 61/638,424, filed on Apr. 25, 2012.

(51) Int. Cl. C07K 16/22

(52) U.S. Cl. CPC C07K 16/22; A61K 2039/505
USPC 424/139.1; 436/501; 530/387.9; 435/7.1; 435/7.9; 435/7.72; 506/9; 435/7.92

(57) ABSTRACT

Compositions and methods for treating and diagnosing acute myocardial infarction are described. The invention also provides a method of treating an individual to prevent or inhibit damage to myocardial tissue from an acute myocardial infarction comprising administering to the individual an antibody to BMP-1-3, or an antibody to BMP-1-4, or a combination of an antibody BMP-1-3 and an antibody to BMP-1-4 prior to AMI.

### CROSS-REFERENCE TO RELATED APPLICATIONS

This application claims priority to U.S. Provisional Application No. 61/638,373, filed Apr. 25, 2012, and to U.S. Provisional Application No. 61/638,424, filed Apr. 25, 2012, the contents of which are incorporated herein in their entireties.

### FIELD OF THE INVENTION

This invention is in the field of heart disease. In particular, the invention provides methods and compositions for treating and diagnosing acute myocardial infarction comprising one or more antibodies to one or more BMP-1 isoforms.

### BACKGROUND OF THE INVENTION

Acute myocardial infarction (“AMI”) is the leading cause of death in developed countries and accounts for 13% of deaths worldwide. Also referred to as “heart attack”, AMI is a type of heart disease that occurs when a coronary artery or vessel becomes occluded resulting in loss of blood supply to myocardial tissue. Myocardial tissue that no longer receives adequate blood (under perfused) dies rapidly and is replaced with poorly functioning or non-functional fibrotic scar tissue, which can expand leading to increased loss of functional myocardial tissue, which in turn can result in a dysfunctional heart. More than one-half of a million people experience a first AMI each year in the United States, and over two hundred thousand people suffering from myocardial infarction die before reaching a hospital.

The intricate relationships among the cellular and acellular components of the heart drive proper heart development, homeostasis, and recovery following pathological injury, such as AMI. Cellular myocytes, fibroblasts, and endothelial cells differentially express and respond to particular extracellular matrix factors that contribute to cell communication and overall cardiac function. The extracellular matrix (“ECM”) facilitates mechanical, electrical, and chemical signals during homeostasis and the developmental process. These signals modulate cellular activities such as cell proliferation, migration, adhesion, and changes in the gene expression. During various physiological cardiac states, different cellular and ECM expression changes take place. See, Bowers et al., J. Molec. Cell. Cardiol., 48: 474-482 (2010). For example, during myocardial infarction myocytes undergo apoptosis, fibroblasts undergo intensive proliferation, vascular density decreases, and an increased expression of collagen I, collagen III, collagen IV, fibronectin, and periostin leads to enhanced fibrosis and diminished cardiac function. These processes have adverse effects on left ventricular function, thus forming a therapeutic basis for use of anti-fibrotic agents to inhibit or reverse such adverse effects. See, for example, Sun et al., Cardiovasc. Res., 46: 250-256 (2000); Jugdutt, Circulation, 108: 1395-1403 (2003); Lopez et al., Am. J. Physiol. Heart. Circ. Physiol., 299: H1-H9 (2010).

Treatments for AMI are typically effective only if implemented rapidly after occlusion of the coronary vessel. Aggressive thrombolytic therapies include drugs that dissolve thrombi (blood clots) or primary angioplasty and stents. Chronic, post-infarction treatments include angiotensin-converting enzyme (“ACE”) inhibitors, beta blockers, diuretics, and calcium channel antagonists, which can reduce aortic pressure, thereby decreasing ventricular remodeling of the left ventricle (LV) that otherwise can expand the size of the infarct leading to more non-functional scar tissue. Open-heart surgical methods include coronary bypass surgery to repair or replace occluded coronary vessels and methods to repair, shrink, or remove the non-functional infarcted region of heart tissue.

Bone morphogenetic protein-1 (“BMP-1”, “BMP1”) was originally isolated from highly purified BMP bovine bone extracts and was originally reported to induce the formation of cartilage in vivo in a subcutaneous (ectopic) bone formation assay (Wozney et al., Science, 242: 1528-1534 (1988)). However, BMP-1 (SEQ ID NO:1) does not share significant amino acid sequence homology with other BMPs, nor does BMP-1 exhibit the characteristic signal peptide, prodomain, carboxy-terminal (mature domain), or cysteine knot found in other BMPs, which are members of the TGFβ super family of growth factors. The erroneous status of BMP-1 within the TGF-β family resulted from flaws in the original bioassay for osteogenesis (Wozney et al., (1988)) in which the cartilage observed in the bioassay appears to have been old growth plate cartilage contaminating the insoluble bone matrix that was misidentified as newly formed tissue (see, Reddi, Science, 271: 463 (1996)). As later shown, BMP-1 clearly does not induce cartilage or bone formation in a standard ectopic bone formation assay. See, for example, international patent publication No. WO 2008/011193 A2.

In fact, BMP-1 was shown to be identical to procollagen C-proteinase, which is a zinc metalloproteinase that cleaves the carboxyl pro-domains of procollagens I, II, and III to produce mature monomers of the major fibrillar collagens I, II, and III (Kessler et al., Science, 271: 360-362 (1996); Li et al., Proc. Natl. Acad. Sci. USA, 93: 5127-5130 (1996)); a step that is essential for the proper assembly of insoluble collagen within the extracellular matrix (ECM) and the formation of fibrous scar tissue as found associated with a variety of organ diseases (Turtle et al., Expert Opin. Ther. Patents, 14(8): 1185-1197 (2004)). In addition to its role in cleaving procollagen, BMP-1 cleaves other ECM macromolecules, including prolysyl oxidase (Panchenko et al., J. Biol. Chem., 271: 7113-7119 (1996)), probiglycan (Scott et al., J. Biol. Chem., 275: 30504-30511 (2000)), and prolaminin-5 (Amano et al., J. Biol. Chem., 275: 22728-22735 (2000)). BMP-1 also releases IGF1 from its binding proteins and other growth factors from their latent complexes (Muir and Greenspan, J. Biol. Chem., 286(49): 41905-41911 (2011)). The BMP-1 protein domain structure comprises an N-terminal prodomain, followed by a conserved protease domain, involved in numerous protein-protein interactions (Bork et al., J. Mol. Biol., 231: 539-545 (1993)). C-terminal to the protease domain are the CUB and EGF domains. The most N-terminal BMP-1 CUB domain (“CUB1”) may cleave chordin, a BMP antagonist which protects BMP-2 and BMP-4 from activation (Petropoulou et al., J. Biol. Chem., 280: 22616-22623 (2005)), while EGF domains bind calcium ion (Ca⁺⁺) and may confer structural rigidity to portions of BMP-1 isoforms (Werner et al. J. Mol. Biol., 296: 1065-1078 (2000)).

The BMP1 gene is related to the Drosophila gene tolloid (“TLD”), which is implicated in the patterning controlled by the decapentaplegic (“DPP”) gene by virtue of its ability to activate TGF-β-like morphogens. The BMP-1 protein is now known to be an essential control point of morphogenesis during the cascade of pattern formation (Ge and Greenspan, Birth Defect Res., 78: 47-68 (2006)). Mice null for the Bmp1 gene are perinatal lethal with failure of ventral body wall closure and persistent gut herniation, likely due to defective ECM and limited disruption of dorsoventral patterning (Suzuki et al., Development, 122: 3587-3595 (1996)). Consistent with a loss of pCP activity, Bmp1-null mice have abnormal collagen fibrils.

BMP-1 is the prototype of a small subgroup of metalloproteinases found in a broad range of species. In mammals, there are four BMP-1/TLD-related (or “BMP-1/TLD-like”) metalloproteinases. The gene encoding BMP-1 also encodes a second, longer proteinase that is encoded by alternatively spliced mRNA. With a domain structure that is essentially identical to TLD, this proteinase was designated mammalian Tolloid (“mTLD”) (Takahara et al., J. Biol. Chem., 269: 32572-32578 (1994)). In addition, there are two genetically distinct mammalian BMP-1/TLD-related proteinases, designated mammalian Tolloid-like 1 and 2 (“mTLL1” and “mTLL2”). The prodomains of BMP-1/TLD-like proteinases must be proteolytically removed by subtilisin-like proprotein convertases (SPCs) (Leighton and Kadler, J. Biol. Chem., 278: 18478-18484 (2003)) to achieve full activity of these proteinases. The role of the prodomain of BMP-1/TLD-like proteinases appears to be in maintaining the BMP-1/TLD-like proteinases in a latent form (Marques et al., Cell, 91: 417-426 (1997); Sieron et al., Biochemistry, 39: 3231-3239 (2000); Leighton and Kadler (2003)).

BMP-1/TLD-related metalloproteinases are responsible for the proteolytic maturation of a number of extracellular proteins related to formation of the extracellular matrix (ECM). These include various collagens, small leucine-rich proteoglycans, SIBLING proteins, lysyl oxidase, laminin-5, and an anti-angiogenic factor from the basement membrane proteoglycan perlecan (Iozzo, Nat. Rev. Mol. Cell. Biol., 6: 646-656 (2005); Greenspan, Top. Curr. Chem., 247: 149-183 (2005); Ge and Greenspan (2006)). BMP-1 is also involved in releasing authentic BMPs from ECM or in activating latent TGF-β family members, such as BMP-4, BMP-11 and GDF-8 (Wolfman et al., Proc. Natl. Acad. Sci. USA, 100: 15842-15846 (2003); Ge et al, Mol. Cell. Biol., 25: 5846-5858 (2005)).

The originally discovered form of BMP-1 is designated as “BMP-1-1” (or “BMP1-1”; SEQ ID NO:1), and other BMP-1 isoforms encoded by splice variant RNA transcripts have been described on the transcriptional level and designated with sequential suffixes: BMP-1-2, BMP-1-3, BMP-1-4, BMP-1-5, BMP-1-6, and BMP-1-7. See, for example, Kessler et al. (1996); Li et al. (1996); Wozney et al. (1988); Janitz et al., J. Mol. Med., 76: 141-146 (1998); Takahara et al. (1994); Hillman et al., Genome Biol., 5(2): R8.1-R8.16 (2004); and Ge and Greenspan, Birth Defect Res., 78: 47-68 (2006). As expected, the BMP-1 isoforms encoded by the splice variant transcripts share a number of domains, including leader peptide, proregion, and protease (catalytic) region. Previously, only the original BMP-1, i.e., BMP-1-1, had been confirmed on the protein level, and the sequences for BMP-1-2 and other BMP-1 isoforms were deduced from nucleotide sequences of the splice variant transcripts, but had not been described at the protein level. More recently, a number of BMP-1 isoforms have been confirmed at the protein level as circulating in the blood of patients with various diseases, such as chronic kidney disease and acute pancreatitis, and in the blood of healthy human individuals (which only contains BMP-1-3). See, for example, international patent publication No. WO 2008/011193 A2; Grgurevic et al., J. Am. Soc. Nephrol., 21: 681-692 (2011). Moreover, the role of BMP-1 in processing procollagen leading to fibrosis and scar tissue in a variety of diseases as well as the discovery of blood profiles comprising individual BMP-1 isoforms in patients of various diseases has made BMP-1 an attractive target for developing new therapies. See, for example, WO 2008/011193 A2, Turtle et al. (2004), and Grgurevic et al. (2011).

Despite the availability of a diversity of drugs and procedures, hundreds of thousands of people die from acute myocardial infarction annually. Clearly, needs remain for new compositions and methods for treating and preventing acute myocardial infarction.

### SUMMARY OF THE INVENTION

The present invention provides new methods and compositions for diagnosis and treatment of acute myocardial infarction (AMI, heart attack) based on the discoveries that human heart tissue contains BMP-1-4; that BMP-1-4 is found circulating in the blood of human subjects that have sustained AMI, but not in healthy individuals; and that BMP-1-3 and BMP-1-4 are therapeutic targets for treating AMI. Accordingly, it is now possible to diagnose AMI by detecting the presence of BMP-1-4 in a sample of blood from a human patient. Moreover, as shown herein, administration of an antibody to BMP-1-3 and/or an antibody to BMP-1-4 is effective to decrease the extent of myocardial tissue damage and even to promote regeneration of functional myocardial tissue in the infarct region of the heart of an individual who has sustained AMI.

In the methods and compositions described herein, the BMP-1-3 protein is the isoform of BMP-1 having the amino acid sequence of SEQ ID NO:2 and the BMP-1-4 protein is the isoform of BMP-1 having the amino acid sequence of SEQ ID NO:3.

In one embodiment of the invention there is provided a method for treating acute myocardial infarction (AMI) in a human subject comprising administering to the subject an antibody to BMP-1-3, or an antibody to BMP-1-4, or a combination of an antibody to BMP-1-3 and an antibody to BMP-1-4. Preferably, the antibodies are neutralizing antibodies.

The invention also provides a method of treating an individual to prevent or inhibit damage to myocardial tissue from an acute myocardial infarction comprising administering to the individual an antibody to BMP-1-3, or an antibody to BMP-1-4, or a combination of an antibody BMP-1-3 and an antibody to BMP-1-4 prior to AMI.

In another embodiment, the present invention provides a method of diagnosing an acute myocardial infarction in a human individual comprising detecting in a sample of blood of the individual the presence of BMP-1-4 having the amino acid sequence of SEQ ID NO:3, or detecting an epitope or a detectable fragment (such as a tryptic fragment) of the BMP-1-4 amino acid sequence.

The diagnostic methods of the present invention for acute myocardial infarction are advantageously carried out using a detector binding molecule capable of binding BMP-1-4, whose presence in a sample of blood that was obtained from an individual indicates that the individual has sustained an acute myocardial infarction. Suitable BMP-1-4 detector binding molecules include antibody molecules that bind BMP-1-4 (including polyclonal antibodies and monoclonal antibodies, genetically engineered antibody molecules, and binding fragments of antibodies such as Fab fragments, F(ab′)₂ fragments, and the like) and aptamers (nucleic acid molecules that have a specific binding affinity for a particular protein) that bind BMP-1-4. An antibody to BMP-1-4 or other BMP-1-4 detector binding molecule may also be associated (covalently or non-covalently) with a detectable label molecule that provides a detectable signal that permits identification of a complex formed by the anti-BMP-1-4 antibody (or other BMP-1-4 detector binding molecule) and the target BMP-1-4 for diagnosing AMI.

A further embodiment of the present invention is a method of diagnosing and treating an individual for acute myocardial infarction comprising:
(a) detecting the presence of BMP-1-4 in a sample of blood from the individual, wherein the presence of BMP-1-4 in the sample indicates that the individual has sustained an acute myocardial infarction;
and
(b) administering to the individual detected as having sustained an acute myocardial infarction in step (a) an antibody to BMP-1-3, an antibody to BMP-1-4, or a combination of an antibody to BMP-1-3 and an antibody to BMP-1-4.

### BRIEF DESCRIPTION OF THE DRAWINGS

FIG. 1 shows diagrams of the domains of full-length (unprocessed) BMP-1-1, BMP-1-3, and BMP-1-4 proteins encoded by BMP1 gene isoforms (alternative spliced products) with indicated common and isoform-specific domains. Domains not drawn to scale. Location of corresponding splice junction within the coding sequence for each isoform is indicated by a gap and bridge between corresponding protein domains. “Leader”=signal peptide sequence. “Prodomain”=N-terminal propeptide domain, which appears to maintain BMP-1 metalloproteinases in a latent form and which must be cleaved to provide the fully active proteinase activity. “Proteinase”=common astacin-like catalytic domain. “CUB”=CUB domains of BMP-1 isoforms, wherein each CUB domain is distinguished serially by a number. “EGF”=calcium-binding epidermal growth factor (EGF)-like domain, wherein each EGF domain is distinguished serially by a number. “ISD”=isoform-specific domain, which is a C-terminal peptide domain specific for each BMP-1 isoform. ISD for the BMP-1-1 isoform protein is a peptide having amino acid residues 703-730 of SEQ ID NO:1. ISD for the BMP-1-3 isoform protein is a peptide having amino acid residues 977-986 of SEQ ID NO:2. ISD for the BMP-1-4 isoform protein is a peptide having amino acid residues 245-302 of SEQ ID NO:3.

FIG. 2 shows a graph of the level (Units/Liter, “U/L”) of creatine kinase myocardial band protein (“CK-MB”) in the blood of rats with ligation-induced acute myocardial infarction (AMI) versus time (“Days”) after surgical ligation of the left coronary artery to induce AMI. Filled diamonds=level of CK-MB in blood of rats with ligation-induced AMI that were treated with monoclonal antibody to BMP-1-3 (“BMP1-3 mAb”). Open squares=level of CK-MB in blood of control rats with ligation-induced AMI that were not treated with BMP-1-3 mAb therapy (“Control”). Asterisk indicates a statistical significance in the level of CK-MB in rats treated with antibody as compared to that in control rats (*p<0.05). See Example 4 for details.

FIG. 3 shows a graph of the level (Units/Liter, “U/L”) of creatine kinase myocardial band protein (“CK-MB”) in the blood of rats with ligation-induced acute myocardial infarction (AMI) versus time (“Days”) after surgical ligation of the left coronary artery to induce AMI. Filled diamonds=level of CK-MB in blood of rats with ligation-induced AMI that were treated with polyclonal antibody to BMP-1-4 (“BMP1-4 Ab”). Open squares=level of CK-MB in the blood of control rats with ligation-induced AMI that were not treated with BMP-1-4 Ab therapy (“Control”). Asterisk indicates a statistical significance in the level of CK-MB in rats treated with antibody as compared to that in control rats (*p<0.05). See Example 5 for details.

FIG. 4 shows a graph of the level (μg/L) of troponin t protein in the blood of rats with ligation-induced acute myocardial infarction (AMI) versus time (Days) after surgical ligation of the left coronary artery to induce AMI. Filled diamonds=level of troponin t in the blood of rats with ligation-induced AMI that were treated with a combination of monoclonal antibody to BMP-1-3 and monoclonal antibody to BMP-1-4 (“BMP1-3 mAb+BMP1-4 mAb”). Open squares=level of troponin t in the blood of control rats with ligation-induced AMI that were not treated with antibody (“Control”). Asterisk indicates a statistical significance in the level of troponin t in rats treated with antibody as compared to that in control rats (*p<0.05). See Example 6 for details.

FIG. 5 shows reconstructed PET scan images of hearts of rats before surgery (“preop”), at one week following ligation surgery to induce AMI (“1 week”), and at one month following surgery to induce AMI (“1 month”) for rats that were treated with BMP-1-3 mAb (“BMP1-3 mAb”) and for control rats with AMI that were not treated with antibody (“control”). Arrows indicate the defect area at one week and one month after surgery for rats that were treated with BMP-1-3 mAb. Restoration of functional myocardial tissue in original infarction region of the heart is clearly indicated after one month in the animals treated with BMP-1-3 mAb whereas loss of functional tissue remains evident after one month in the heart of untreated control animals. See Example 8 for details.

FIG. 6 shows micrographs from a histological analysis of the heart muscle in rats following coronary artery ligation with and without BMP-1-3 monoclonal antibody therapy. FIG. 6A shows a heart section from the infarcted area of the heart of a rat at one week after ligation of the left coronary artery to induce acute myocardial infarction (AMI) in the absence of antibody therapy (magnification 4×). Rectangle in FIG. 6A is magnified in FIG. 6B. FIG. 6B shows Sirius red staining of tissue of rectangle area in FIG. 6A (at 20× magnification) indicating early collagen deposition. See, arrows in FIG. 6B. FIG. 6C shows a section of myocardial tissue from an untreated rat with AMI stained with hematoxylin and eosin revealing residual fibrotic scar tissue after 1 month surrounded by damaged myocardial fibers. See arrow in FIG. 6C. FIG. 6D shows a heart section from the infarcted area of the heart of a rat treated with BMP-1-3 mAb (15 μg/kg) prior to ligation of the left coronary artery to induce AMI and then treated with BMP-1-3 mAb every day during the first week after surgery. The fibrotic area following AMI was significantly smaller than that observed in control rats. See, arrow in FIG. 6D. FIG. 6E shows a higher magnification of the area indicated by arrow in FIG. 6D revealing spots of new regenerative muscle fibers. See, arrows in FIG. 6E. FIG. 6F shows a more detailed view of the area indicated by arrows in FIG. 6D revealing newly formed muscle fibers and surrounding cells with fibrous tissue that is less dense than that observed in control rats. See Example 9 for details.

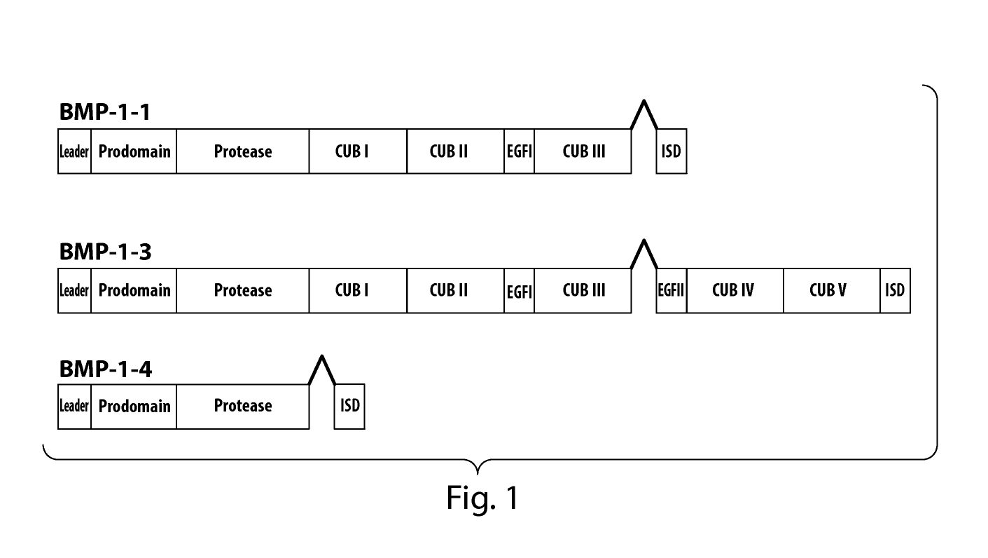

### DETAILED DESCRIPTION OF THE INVENTION

The invention described herein is based on the discovery that both BMP-1-3 and BMP-1-4 proteins are present in the blood of adult human individuals that have sustained acute myocardial infarction (“AMI”, “heart attack”) and that these two BMP-1 isoforms are also therapeutic targets for treating AMI.

In order that the invention may be fully understood the following terms are defined.

The amino acid sequence of the full-length (unprocessed) BMP-1-1 protein described herein has the amino acid sequence:

(SEQ ID NO: 1)
MPGVARLPLL LGLLLLPRPG RPLDLADYTY DLAEEDDSEP
LNYKDPCKAA AFLGDIALDE EDLRAFQVQQ AVDLRRHTAR
KSSIKAAVPG NTSTPSCQST NGQPQRGACG RWRGRSRSRR
AATSRPERVW PDGVIPFVIG GNFTGSQRAV FRQAMRHWEK
HTCVTFLERT DEDSYIVFTY RPCGCCSYVG RRGGGPQAIS
IGKNCDKFGI VVHELGHVVG FWHEHTRPDR DRHVSIVREN
IQPGQEYNFL KMEPQEVESL GETYDFDSIM HYARNTFSRG
IFLDTIVPKY EVNGVKPPIG QRTRLSKGDI AQARKLYKCP
ACGETLQDST GNFSSPEYPN GYSAHMHCVW RISVTPGEKI
ILNFTSLDLY RSRLCWYDYV EVRDGFWRKA PLRGRFCGSK
LPEPIVSTDS RLWVEFRSSS NWVGKGFFAV YEAICGGDVK
KDYGHIQSPN YPDDYRPSKV CIWRIQVSEG FHVGLTFQSF
EIERHDSCAY DYLEVRDGHS ESSTLIGRYC GYEKPDDIKS
TSSRLWLKFV SDGSINKAGF AVNFFKEVDE CSRPNRGGCE
QRCLNTLGSY KCSCDPGYEL APDKRRCEAA CGGFLTKLNG
SITSPGWPKE YPPNKNCIWQ LVAPTQYRIS LQFDFFETEG
NDVCKYDFVE VRSGLTADSK LHGKFCGSEK PEVITSQYNN
MRVEFKSDNT VSKKGFKAHF FSEKRPALQP PRGRPHQLKF
RVQKRNRTPQ.

The amino acid sequence of the full-length (unprocessed) BMP-1-3 protein described herein has the amino acid sequence:

(SEQ ID NO: 2)
MPGVARLPLL LGLLLLPRPG RPLDLADYTY DLAEEDDSEP
LNYKDPCKAA AFLGDIALDE EDLRAFQVQQ AVDLRRHTAR
KSSIKAAVPG NTSTPSCQST NGQPQRGACG RWRGRSRSRR
AATSRPERVW PDGVIPFVIG GNFTGSQRAV FRQAMRHWEK
HTCVTFLERT DEDSYIVFTY RPCGCCSYVG RRGGGPQAIS
IGKNCDKFGI VVHELGHVVG FWHEHTRPDR DRHVSIVREN
IQPGQEYNFL KMEPQEVESL GETYDFDSIM HYARNTFSRG
IFLDTIVPKY EVNGVKPPIG QRTRLSKGDI AQARKLYKCP
ACGETLQDST GNFSSPEYPN GYSAHMHCVW RISVTPGEKI
ILNFTSLDLY RSRLCWYDYV EVRDGFWRKA PLRGRFCGSK
LPEPIVSTDS RLWVEFRSSS NWVGKGFFAV YEAICGGDVK
KDYGHIQSPN YPDDYRPSKV CIWRIQVSEG FHVGLTFQSF
EIERHDSCAY DYLEVRDGHS ESSTLIGRYC GYEKPDDIKS
TSSRLWLKFV SDGSINKAGF AVNFFKEVDE CSRPNRGGCE
QRCLNTLGSY KCSCDPGYEL APDKRRCEAA CGGFLTKLNG
SITSPGWPKE YPPNKNCIWQ LVAPTQYRIS LQFDFFETEG
NDVCKYDFVE VRSGLTADSK LHGKFCGSEK PEVITSQYNN
MRVEFKSDNT VSKKGFKAHF FSDKDECSKD NGGCQQDCVN
TFGSYECQCR SGFVLHDNKH DCKEAGCDHK VTSTSGTITS
PNWPDKYPSK KECTWAISST PGHRVKLTFM EMDIESQPEC
AYDHLEVFDG RDAKAPVLGR FCGSKKPEPV LATGSRMFLR
FYSDNSVQRK GFQASHATEC GGQVRADVKT KDLYSHAQFG
DNNYPGGVDC EWVIVAEEGY GVELVFQTFE VEEETDCGYD
YMELFDGYDS TAPRLGRYCG SGPPEEVYSA GDSVLVKFHS
DDTITKKGFH LRYTSTKFQD TLHSRK.

The amino acid sequence of the full-length (unprocessed) BMP-1-4 protein described herein has the amino acid sequence:

(SEQ ID NO: 3)
MPGVARLPLL LGLLLLPRPG RPLDLADYTY DLAEEDDSEP
LNYKDPCKAA AFLGDIALDE EDLRAFQVQQ AVDLRRHTAR
KSSIKAAVPG NTSTPSCQST NGQPQRGACG RWRGRSRSRR
AATSRPERVW PDGVIPFVIG GNFTGSQRAV FRQAMRHWEK
HTCVTFLERT DEDSYIVFTY RPCGCCSYVG RRGGGPQAIS
IGKNCDKFGI VVHELGHVVG FWHEHTRPDR DRHVSIVREN
IQPGVLHSSL LLLSCGSRNG ASFPCSLESS THQALCWTGL
FLRPSPFPRL PLAAPRTLRA GV.

Unless indicated otherwise, when the terms “about” and “approximately” are used in combination with an amount, number, or value, then that combination describes the recited amount, number, or value alone as well as the amount, number, or value plus or minus 10% of that amount, number, or value. By way of non-limiting example, the phrases “about 40%” and “approximately 40%” disclose both “40%” and “from 36% to 44%, inclusive”.

“Antibody” or “antibody molecule”, as used and understood herein, refers to a specific binding member that is a protein molecule or portion thereof, whether produced naturally, synthetically, or semi-synthetically, that possesses an antigen binding domain comprising an immunoglobulin light chain variable region or domain (V_{L}) or portion thereof, an immunoglobulin heavy chain variable region or domain (V_{H}) or portion thereof, or a combination thereof, and that binds a specific target molecule (antigen). The term “antibody” also encompasses any polypeptide or protein molecule that has an antigen binding domain that is identical, or homologous to, an antigen-binding domain of an immunoglobulin. Antibodies may be “polyclonal”, i.e., a population of antigen-binding molecules produced in a multiplicity of different cells and which consequently bind to different sites on an antigen, or “monoclonal”, i.e., a population of identical antigen-binding molecules produce from a single cell line that bind to only one site on an antigen (i.e., the same epitope of an antigen). Examples of an antibody molecule, as used and understood herein, include any of the well-known classes of immunoglobulins (e.g., IgG, IgM, IgA, IgE, IgD) and their isotypes; fragments of immunoglobulins that comprise an antigen binding domain, such as Fab or F(ab′)₂ molecules; single chain antibody (scFv) molecules; double scFv molecules; single domain antibody (dAb) molecules, which possess a functional antigen-binding domain that comprises only three CDRs of a single heavy chain variable domain that can bind to antigen in a 1:1 ratio without a corresponding light chain variable domain (see, e.g., Ward et al., Nature, 341: 544-546 (1989); international publication No. WO 90/05144; Hamers-Casterman et al., Nature, 363: 446-448 (1993), Muyldermans et al., Protein Eng., 7: 1129-1135 (1994)); Fd molecules (consisting of an antibody VH region linked to antibody heavy chain constant domains CH1, CH2, CH3, and, optionally, CH4); diabody molecules; and fusion proteins comprising such molecules. Diabodies are formed by association of two diabody monomers, which form a dimer that contains two complete antigen binding domains wherein each binding domain is itself formed by the intermolecular association of a region from each of the two monomers (see, e.g., Holliger et al., Proc. Natl. Acad. Sci. USA, 90: 6444-6448 (1993)). An antibody to BMP-1-3 or BMP-1-4 that is useful in the compositions and methods described herein also may be a bispecific antibody that comprises an antigen-binding domain that specifically binds a molecule of BMP-1-3 and another antigen-binding domain that specifically binds a molecule of BMP-1-4. An antibody molecule to BMP-1-3 or BMP-1-4 that may be used in the compositions and methods described herein also may be a dual variable domain (DVD) binding proteins (see, for example, international patent publication No. WO 2007/024715) that comprises an antigen-binding domain that specifically binds BMP-1-3 or an antigen-binding domain that specifically binds BMP-1-4 or that comprises an antigen-binding domain that specifically binds a molecule of BMP-1-3 and another antigen-binding domain that specifically binds a molecule of BMP-1-4. All of the above molecules are binding proteins useful in methods described herein because they comprise a functional binding domain for BMP-1-3 and/or a functional binding domain for BMP-1-4. Antibodies binding to BMP-1-3 or BMP-1-4 will alternatively be referred to herein as “BMP-1-3 antibodies” or “BMP-1-4 antibodies”, respectively, and also “anti-BMP-1-3 antibodies” and “anti-BMP-1-4 antibodies”, respectively.

An “isolated antibody” is intended to refer to an antibody that is substantially free of other antibody molecules and antibody fragments having different antigenic specificities (e.g., an isolated antibody that specifically binds a particular BMP-1 isoform, such as BMP-1-3 or BMP-1-4, is substantially free of antibody molecules that specifically bind antigens other than the particular BMP-1 isoform). An “isolated antibody” that specifically binds a particular BMP-1-3 may, however, have cross-reactivity to other antigens, such as a BM-1-3 from other species. Moreover, an isolated antibody may be substantially free of other cellular material and/or chemicals.

The term “monoclonal antibody” or “mAb” refers to an antibody obtained from a population of substantially homogeneous antibody molecules, i.e., the individual antibody molecules comprising the population are identical except for possible naturally occurring mutations that may be present in minor amounts. Monoclonal antibodies are highly specific, being directed against a single antigen. Furthermore, in contrast to polyclonal antibody preparations that typically include different antibody molecules directed against different antigenic determinants (epitopes) of an antigen, each mAb molecule is directed against a single epitope of the antigen. The modifier “monoclonal” is not to be construed as requiring production of the antibody by any particular method.

The term “human antibody” includes antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs and in particular CDR-H3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created, or isolated by recombinant means, such as antibodies expressed using a recombinant expression vector transfected into a host cell, antibodies isolated from a recombinant, combinatorial human antibody library (Hoogenboom, Trends Biotechnol., 15: 62-70 (1997); Azzazy and Highsmith, Clin. Biochem., 35: 425-445 (2002); Gavilondo and Larrick, BioTechniques, 29: 128-145 (2000); Hoogenboom and Chames, Immunol. Today, 21: 371-378 (2000)), antibodies isolated from an animal (e.g., a mouse) that is transgenic for human immunoglobulin genes (see, e.g., Taylor et al., Nucl. Acids Res., 20: 6287-6295 (1992); Kellermann and Green, Curr. Opin. Biotechnol., 13: 593-597 (2002); Little et al., Immunol. Today, 21: 364-370 (2000)); or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies that comprise heavy and light chain variable region sequences from one species and constant region sequences from another species, such as antibodies having murine heavy and light chain variable regions linked to human constant regions.

The term “CDR” refers to the complementarity determining region within antibody variable regions. There are three CDRs in each antibody variable region and are designated “CDR1”, “CDR2”, and “CDR3”, wherein by convention as adopted herein “CDR1” refers to the most N-terminal proximal of the three CDRs within an antibody variable region and “CDR3” refers to the most C-terminal proximal of the three CDRs within an antibody variable region. The CDRs within an antibody heavy chain variable region (VH) are designated “CDR-H1”, “CDR-H2”, and “CDR-H3”, and the CDRs with an antibody light chain variable region (VL) are designated “CDR-L1”, “CDR-L2”, and “CDR-L3”.

The term “CDR set” as used herein refers to a group of three CDRs that occur in a single variable region capable of binding a particular epitope of an antigen molecule. The exact boundaries of these CDRs have been defined differently according to different numbering systems. The system described by Kabat (Kabat et al., Sequences of Proteins of Immunological Interest (National Institutes of Health, Bethesda, Md. (1987) and (1991)); Kabat et al., Sequences of Proteins of Immunological Interest, Fifth Edition, U.S. Department of Health and Human Services, NIH Publication No. 91-3242 (1991)) is the most widely used numbering system. The Kabat numbering system provides a residue numbering system for the residues within a variable region and provides precise residue boundaries defining the three CDRs. Other numbering systems were later devised, but the Kabat numbering system is still the most widely used numbering system for assigning positions of residues within antibody variable regions and for identifying the amino acid sequences for each of the CDRs within an antibody variable region.

The growth and analysis of extensive public databases of amino acid sequences of variable heavy and light regions over the past twenty years have led to the understanding of the typical boundaries between framework regions (FR) and CDR sequences within variable region sequences and enabled persons skilled in this art to accurately determine the CDRs according to Kabat numbering, Chothia numbering, or other systems. See, e.g., Martin, “Protein Sequence and Structure Analysis of Antibody Variable Domains,” Chapter 31, In Antibody Engineering, (Kontermann and Dübel, eds.) (Springer-Verlag, Berlin, 2001), especially pages 432-433. A useful method of determining the amino acid sequences of Kabat CDRs within the amino acid sequences of variable heavy (VH) and variable light (VL) regions is provided below:

To identify a CDR-L1 amino acid sequence:

Starts approximately 24 amino acid residues from the amino terminus of the VL region;
Residue before the CDR-L1 sequence is always cysteine (C);
Residue after the CDR-L1 sequence is always a tryptophan (W) residue, typically Trp-Tyr-Gln (W-Y-Q), but also Trp-Leu-Gln (W-L-Q), Trp-Phe-Gln (W-F-Q), and Trp-Tyr-Leu (W-Y-L);
Length is typically 10 to 17 amino acid residues.

To identify a CDR-L2 amino acid sequence:

Starts always 16 residues after the end of CDR-L1;
Residues before the CDR-L2 sequence are generally Ile-Tyr (I-Y), but also Val-Tyr (V-Y), Ile-Lys (I-K), and Ile-Phe (I-F);
Length is always 7 amino acid residues.

To identify a CDR-L3 amino acid sequence:

Starts always 33 amino acids after the end of CDR-L2;
Residue before the CDR-L3 amino acid sequence is always a cysteine (C);
Residues after the CDR-L3 sequence are always Phe-Gly-X-Gly (F-G-X-G) (SEQ ID NO:4), where X is any amino acid;
Length is typically 7 to 11 amino acid residues.

To identify a CDR-H1 amino acid sequence:

Starts approximately 31 amino acid residues from amino terminus of VH region and always 9 residues after a cysteine (C);
Residues before the CDR-H1 sequence are always Cys-X-X-X-X-X-X-X-X (SEQ ID NO:5), where X is any amino acid;
Residue after CDR-H1 sequence is always a Trp (W), typically Trp-Val (W-V), but also Trp-Ile (W-I), and Trp-Ala (W-A);
Length is typically 5 to 7 amino acid residues.

To identify a CDR-H2 amino acid sequence:

Starts always 15 amino acid residues after the end of CDR-H1;
Residues before CDR-H2 sequence are typically Leu-Glu-Trp-Ile-Gly (L-E-W-I-G) (SEQ ID NO:6), but other variations also;
Residues after CDR-H2 sequence are usually Lys/Arg-Leu/Ile/Val/Phe/Thr/Ala-Thr/Ser/Ile/Ala (K/R-L/I/V/F/T/A-T/S/I/A);
Length is typically 16 to 19 amino acid residues.

To identify a CDR-H3 amino acid sequence:

Starts always 33 amino acid residues after the end of CDR-H2 and always 3 residues after a cysteine (C);
Residues before the CDR-H3 sequence are always Cys-X-X (C-X-X), where X is any amino acid, typically Cys-Ala-Arg (C-A-R);
Residues after the CDR-H3 sequence are always Trp-Gly-X-Gly (W-G-X-G) (SEQ ID NO:7), where X is any amino acid;
Length is typically 3 to 25 amino acid residues.

The term “CDR-grafted antibody” refers to an antibody molecule that comprises heavy and light chain variable region sequences from one species but in which the sequences of one or more of the CDR regions in the VH and/or VL regions are replaced with CDR sequences of another species, such as antibodies having human heavy and light chain variable regions in which one or more of the human CDRs (e.g., CDR3) has been replaced with murine CDR sequences. Methods for grafting CDRs of an antibody of one species into the variable domains of an antibody of another species are well known in the art. See, for example, Jones et al., Nature, 321: 522-525 (1986); Riechmann et al., Nature, 332: 323-327 (1988); Verhoeyen et al., Science, 239: 1534-1536 (1988); and Queen et al., Proc. Natl. Acad. Sci. USA, 86: 10029-10033 (1989).

“Circulate” and “circulating” describe anything that travels or is otherwise transported through the vascular system of an individual.

The terms “disorder” and “disease” are synonymous and refer to any pathological condition, irrespective of cause or etiological agent. A “defect” in a tissue refers to a site of abnormal or deficient tissue growth. A “disease” or “disorder” may be characterized by one or more “defects” in one or more tissues. The disease (or disorder) of interest to this invention is acute myocardial infarction (“AMI”, “heart attack”).

As used herein, the terms “treatment” and “treating” refer to any regimen that alleviates one or more symptoms or manifestations of a disease or disorder, that inhibits progression of a disease or disorder, that arrests progression or reverses progression (causes regression) of a disease or disorder, or that prevents onset of a disease or disorder. The term “treatment” includes prophylaxis (prevention) of one or more symptoms or manifestations of a disease, including ameliorating or inhibiting the extent of a symptom or manifestation that would otherwise characterize the disease in the absence of the treatment.

A “therapeutically effective amount” is an amount of a compound (for example, an antibody to BMP-1-3, an antibody to BMP-1-4, or a combination thereof) that inhibits, totally or partially, the progression of a disease; that alleviates, at least partially, one or more symptoms of the disease; or that enhances or catalyzes the therapeutic or otherwise beneficial effects of another compound employed for treating a disease. A therapeutically effective amount can also be an amount that is prophylactically effective. The amount that is therapeutically effective will depend upon the patient's size and gender, the disease to be treated, the severity of the disease, and the result sought. For a given human individual, a therapeutically effective amount can be determined by methods known to those of skill in the art.

The term “isolated” when used to describe the various proteins or polypeptides disclosed herein, means a protein or polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous species. An isolated protein or polypeptide includes a protein or polypeptide in situ within recombinant cells engineered to express it, since at least one component of the protein's or polypeptide's natural environment will not be present. Ordinarily, however, an isolated protein or polypeptide will be prepared by at least one purification step.

A composition or method described herein as “comprising” one or more named elements or steps is open-ended, meaning that the named elements or steps are essential, but other elements or steps may be added within the scope of the composition or method. To avoid prolixity, it is also understood that any composition or method described herein as “comprising” (or “which comprises”) one or more named elements or steps also describes the corresponding, more limited, composition or method “consisting essentially of” (or “which consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and close-ended composition or method “consisting of” (or “which consists of”) the named elements or steps to the exclusion of any other unnamed element or step. In any composition or method disclosed herein, known or disclosed equivalents of any named essential element or step may be substituted for that element or step.

Unless indicated otherwise, the meaning of other terms is the same as understood and used by persons in the art, including the fields of medicine, immunology, biochemistry, molecular biology, and tissue regeneration.

The invention is based on the discovery that BMP-1-4 is present in the blood of human individuals that have sustained acute myocardial infarction (AMI) but not in the blood of healthy individuals. The BMP-1-3 isoform protein, which is present circulating in the blood of healthy individuals, is also present in the blood of individuals that have sustained AMI. Accordingly, BMP-1-4 is useful as a new blood biological marker (biomarker) for AMI.

As previously shown, BMP-1 isoform proteins can be detected in samples of blood by analyzing the blood for the presence of one or more peptides (for example, tryptic peptides) that are unique to a particular BMP-1 isoform. See, for example, WO 2008/011193; Grgurevic et al. (2011). As shown in Example 1, below, this type of peptide analysis demonstrated for the first time the existence of the BMP-1-4 isoform protein at the protein level in humans. Moreover, the BMP-1-4 protein was detected in the blood of patients that had sustained an acute myocardial infarction, but not in the blood of healthy volunteers. BMP-1-4 and BMP1-3 were localized in the heart of the developing human embryo and in sections of heart of human individuals that had sustained AMI using antibody to BMP-1-3 and BMP-1-4 (data not shown). Thus, BMP-1-4 is normally expressed in normal heart tissue, but appears in the blood of individuals that have sustained AMI. The appearance of BMP-1-4 in the blood of human individuals who have sustained AMI is also correlated with the appearance of plasma troponin t (“Tn-T”) and elevated levels of creatine kinase myocardial band (CK-MB) in the blood of individuals that have sustained AMI. Accordingly, BMP-1-4 is useful as a blood biomarker for AMI.

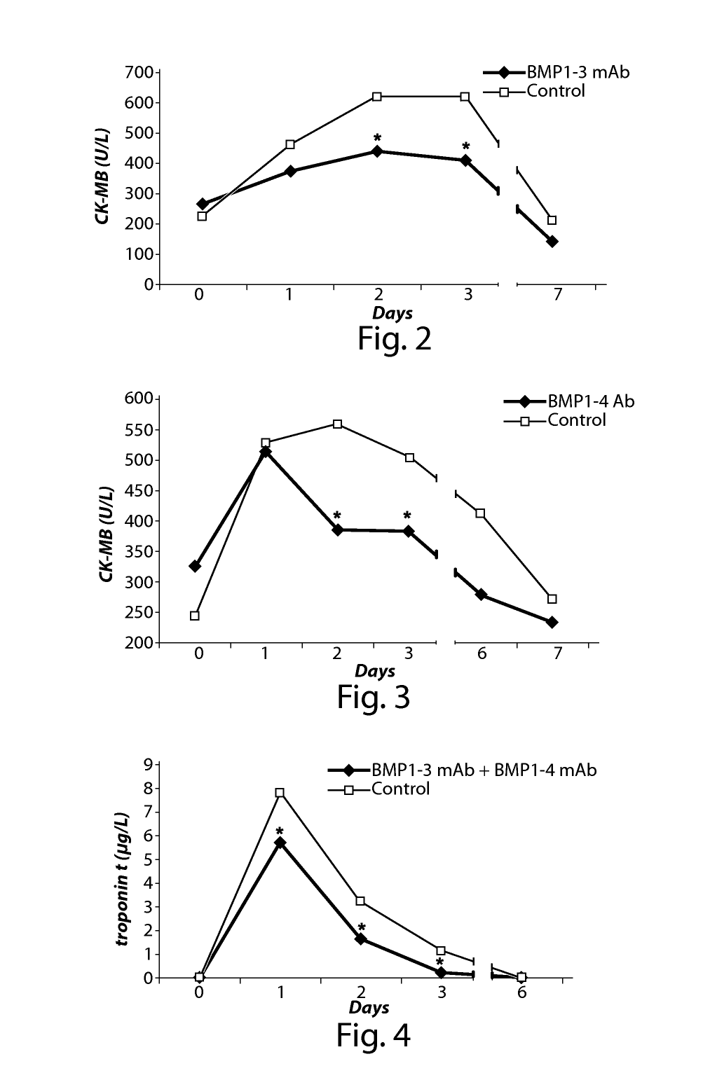

The findings described herein that BMP-1-4 appears in the blood of individuals that have sustained AMI and that BMP-1-4 is localized in healthy heart tissue, and prior findings indicating that BMP-1-1 and BMP-1-3 promote fibrosis and scar tissue in other organs (see, for example, Turtle et al. (2004), WO 2008/011193, Grgurevic et al. (2011)), led the inventors to investigate whether either or both of BMP-1-3 and BMP-1-4 could possibly be useful as therapeutic targets for treating AMI. The results of studies using a standard rat model for AMI described in the Examples below, clearly show that both BMP-1-3 and BMP-1-4 are therapeutic targets for treating AMI. For example, administration of a monoclonal antibody to BMP-1-3 (“BMP-1-3 mAb”) to rats with AMI resulted in a significantly lower elevation of plasma levels of the biomarker CK-MB as compared to that in untreated control rats with AMI. See, Example 4 and FIG. 2. Administration to rats with AMI of an antibody to BMP-1-4 also resulted in a significantly lower elevation of plasma levels of the CK-MB as compared to that in untreated control rats with AMI. See, Example 5 and FIG. 3. Administration of a combination of BMP-1-3 mAb and BMP-1-4 mAb to rats with AMI also resulted in significantly lower levels of plasma levels of the troponin t (“Tn-T”) than in untreated control rats. See, Example 6 and FIG. 4.

Moreover, therapeutic efficacy of administering BMP-1-3 mAb to rats with AMI also was indicated by echocardiography assessment of heart dimensions and function that revealed significantly higher interventricular septal dimensions at diastole (IVSd) and at systole (IVSs), significantly lower left ventricular internal dimension at systole (LVIDs), significantly lower left ventricular posterior wall dimension at systole (LVPWs), significantly higher ejection fraction (EF), and significantly higher fractional shortening (ES) as compared to those in untreated control rats. In fact, the heart dimensions and functions of antibody-treated rats were similar to those of sham rats without AMI. See Example 7 and Table 1.

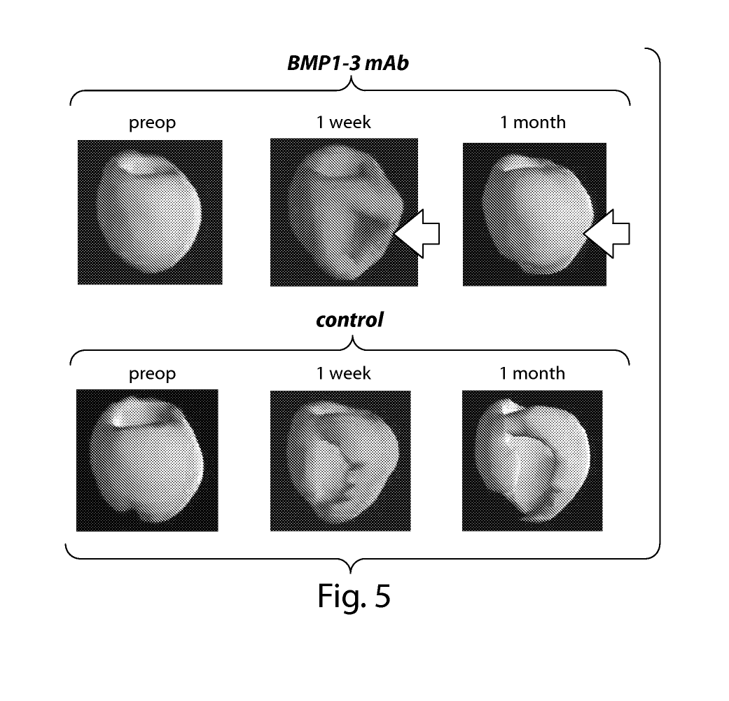

Treatment with BMP-1-3 mAb was also shown to promote a higher quality of scar tissue and functional myocardial tissue in an infarct region of the heart after AMI. This therapeutic effect of antibody treatment was dramatically shown by monitoring an infarcted region of the heart over the course of a month using positron emission tomography (PET) as described in Example 8. As shown in FIG. 5, the hearts of rats with AMI were PET scanned prior to surgical induction of AMI (“preop”), at one week after surgery (“1 week”), and at one month after surgery (“1 month”). The PET scan images of the heart of a rat that was treated with BMP-1-3 mAb and that of an untreated control rat clearly show non-functional tissue in the infarct regions at one week after surgery, although the infarct region of the untreated control rat appears to be more pronounced than that of the treated rat. See images of hearts at “1 week” in FIG. 5. However, one month after surgical induction of AMI, the PET scan images revealed a dramatic difference in the quality of tissue that is generated in the original infarct region of the hearts of the treated and untreated animals. In particular, the PET scan image of the heart of the rat that received BMP-1-3 mAb treatment showed a substantial restoration of functional myocardial tissue in the infarct region, whereas the non-functional tissue in the infarct region of the untreated control rat was clearly retained and even more pronounced than at one week. See images of hearts at “1 month” in FIG. 5. The results show that the repaired tissue in the heart of the rat that received antibody therapy was clearly of a higher quality and more functional than the repair tissue generated in the heart of the untreated control rat.

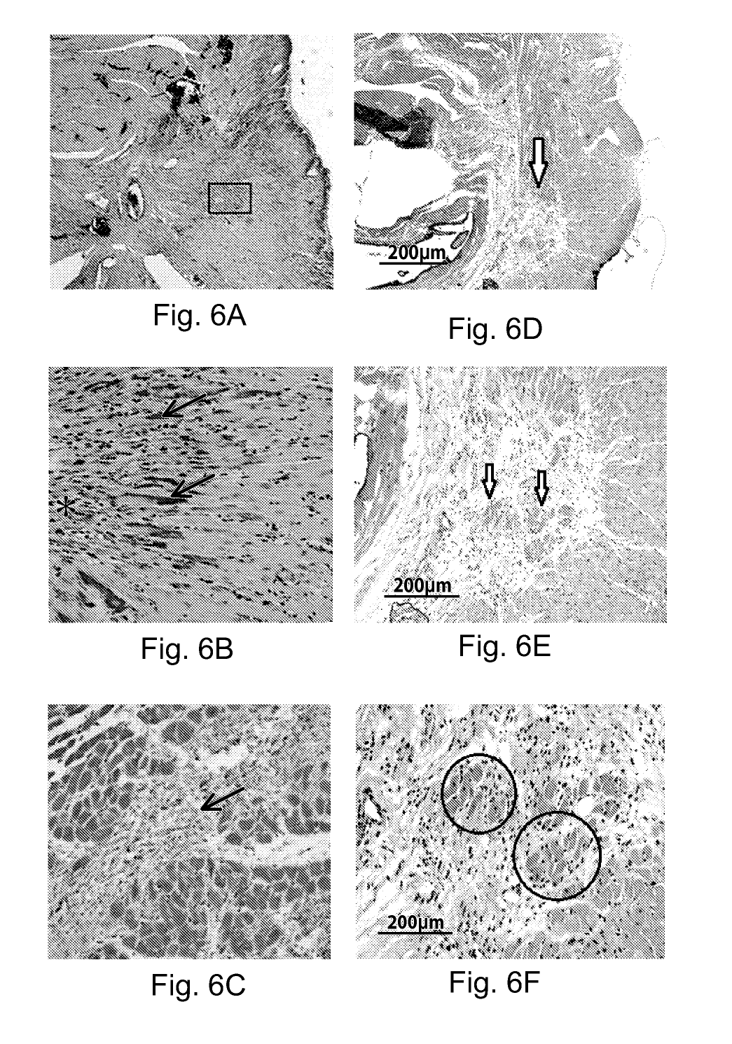

Histological analysis of myocardial tissue from hearts of untreated control rats with AMI and from rats with AMI that were treated with BMP-1-3 mAb also showed a beneficial effect of treatment with BMP-1-3 antibody as described in Example 9, below. In particular, at one week following surgical ligation of the left coronary artery to induce AMI, Sirius red staining of myocardial tissue from untreated control rats revealed early collagen deposition (see, FIG. 6B). At one month after surgery, Sirius red staining of myocardial tissue from untreated control rats revealed residual fibrotic scar tissue that was clearly surrounded by damaged myocardial fibers (see, FIG. 6C). In contrast, the fibrotic area at one week following AMI was significantly smaller in myocardial tissue of rats treated with BMP-1-3 mAb (see, FIG. 6D) compared to that of untreated control rats. Higher magnification of the tissue shown in FIG. 6D revealed spots of newly formed muscle fibers (see, FIG. 6E) and surrounding cells with fibrous tissue that was clearly less dense that observed in untreated control rats (see, FIG. 6F).

Any of a variety methods known in the art may be employed to produce polyclonal or monoclonal antibody molecules that specifically bind a specific BMP-1 isoform of interest (such as BMP-1-3 or BMP-1-4) or a portion of the specific BMP-1 isoform of interest comprising at least one epitope (i.e., the specific antibody binding site) of the specific BMP-1 isoform.

Polyclonal antibodies may be produced using standard methods known in the art in which an antigen (for example, BMP-1-3, BMP-1-4, or peptide comprising an epitope of BMP-1-3 or BMP-1-4) is administered to an animal under conditions that elicit an immune response by the animal resulting in the production of antibodies to the antigen. Typically, such polyclonal antibodies are produced in the blood of an animal and can be isolated in the serum portion of the blood (antiserum). Further purification may provide a polyclonal antibody preparation of enhanced purity or the isolation of specific classes of antibodies from the antiserum.

Preferred antibody molecules for use in the compositions and methods described herein are monoclonal antibodies (mAbs) to BMP-1-3 and to BMP-1-4. Monoclonal antibodies can be prepared using standard hybridoma technology available in the art. Such techniques are described in standard laboratory manuals of the art. See, for example, Harlowe et al., Antibodies: A Laboratory Manual, Second Edition (Cold Spring Harbor Laboratory Press, 1988); -Monoclonal Antibodies and T-Cell Hybridomas (Elsevier, New York, 1981); incorporated herein by reference. Antibodies to BMP-1-3 and BMP-1-4 also can be generated using any of a number of other methods available in the art. For example, antibodies to BMP-1-3 and BMP-1-4 may be generated from single, isolated lymphocytes using a selected lymphocyte antibody method (SLAM). See, for example, U.S. Pat. No. 5,627,052; international publication No. WO 92/02551; Babcook et al., Proc. Natl. Acad. Sci. USA, 93: 7843-7848 (1996); incorporated herein by reference. Antibodies to BMP-1-3 and BMP-1-4 can also be prepared using a transgenic animal that comprises all or a portion of a human immunoglobulin locus that will produce human antibody when the transgenic animal is immunized with BMP-1-3 or BMP-1-4 protein or peptide fragment thereof. See, for example, Green et al., Nature Genetics, 7:13-21 (1994); U.S. Pat. No. 5,916,771; international patent publication No. WO 91/10741; incorporated herein by reference. Other methods for producing BMP-1-3 and BMP-1-4 antibodies useful in the compositions and methods described herein include, without limitation, phage display methods (for example, Brinkmann et al., J. Immunol. Methods, 182: 41-50 (1995); Ames et al., J. Immunol. Methods, 184: 177-186 (1995), Kettleborough et al., Eur. J. Immunol., 24:952-958 (1994)) incorporated herein by reference), yeast display methods (see, for example, U.S. Pat. No. 6,699,658, incorporated herein by reference), and expression of an antibody library as an RNA-protein fusion (see, for example, international patent publication No. WO 98/31700, incorporated herein by reference).

Preferably, a BMP-1-3 mAb is produced using a peptide immunogen that has the amino acid sequence of R-Y-T-S-T-K-F-Q-D-T-L-H-S-R-K (amino acid residues 972-986 of SEQ ID NO:2). A particularly preferred BMP-1-3 mAb, designated ______, is produced by a hybridoma cell line that was prepared on order by ProMab (Richmond, Calif., USA) and that was deposited under the Budapest Treaty in the Leibniz-Institut DSMZ-Deutsche Sammlung von Mikrooganismen and Zellkulturen GmbH (“DSMZ”) on Apr. 24, 2013 (accession no. ______).

Preferably, a BMP-1-4 mAb is produced using a peptide immunogen that has the amino acid sequence of C-G-S-R-N-G-A-S-F-P-S-S-L-E-S-S-T-H-Q-A (SEQ ID NO:8). A BMP-1-4 mAb, designated ______, has been produced on order by ProMab (Richmond, Calif., USA).

A rodent hybridoma cell line that produces a monoclonal antibody (“mAb”) is a ready source of DNA that encodes the constant and variable regions of the mAb molecule. Especially useful is the isolation and sequence determination of DNA encoding the individual complementarity determining regions (“CDRs”) and framework regions (“FRs”) of a BMP-1-3 mAb or BMP-1-4 mAb. Isolated or synthesized DNA encoding the individual CDRs, FRs, and/or portions thereof, of a rodent BMP-1-3 mAb or BMP-1-4 mAb can be readily employed in standard methods for producing any of a variety of other recombinant antibody molecules that bind BMP-1-3 or BMP-1-4. Such recombinant antibody molecules include, but are not limited to, CDR-grafted antibody molecules; chimeric antibodies, humanized antibodies; affinity matured humanized antibodies; single chain antibody (“scFv”) molecules; double scFv molecules; diabody molecules; bispecific antibodies that bind either or both BMP-1-3 or BMP-1-4; and dual variable domain immunoglobulin binding proteins that bind either or both BMP-1-3 and BMP-1-4. A particularly preferred recombinant antibody is a humanized antibody, which binds the same antigen (BMP-1-3 or BMP-1-4) as the original rodent mAb, but is less immunogenic when injected into humans. See, for example, U.S. Pat. No. 5,693,762; Queen et al. (1989); European Patent No. 0 239 400 B1.

Preferably, an antibody to BMP-1-3 and BMP-1-4 used in the methods and compositions of the invention for treating acute myocardial infarction is a neutralizing antibody as demonstrated by the ability of the antibody to inhibit BMP-1-3 or BMP-1-4 mediated cleavage of procollagen in vitro (see, for example, Kessler et al. (1996); Li et al. (1996); Garrigue-Antar et al., J. Biol. Chem., 276(28): 26237-26242 (2001); Hartigan et al., J. Biol. Chem., 278(20):18045-18049 (2003)); by the ability of the antibody to inhibit BMP-1-3 or BMP-1-4 mediated cleavage of dentin matrix protein 1 (DMP-1) in vitro (see, for example, Qin et al., J. Biol. Chem., 278(36): 34700-34708 (2003); Steiglitz et al., J. Biol. Chem., 279(2): 980-986 (2004)); or by the ability of the antibody to inhibit the extent of damage to myocardial tissue in a rat model of acute myocardial infarction (see, review of rat model in Zornoff et al., Arq. Bras. Cardiol., 93(3): 403-408 (2009)).

A method for treating an individual for acute myocardial infarction (AMI) according to the invention comprises the step of administering to the individual an antibody to BMP-1-3, an antibody to BMP-1-4, or a combination of antibody to BMP-1-3 and antibody to BMP-1-4. Preferably, an antibody molecule used for treating a human individual for AMI possesses regions and domains that are those of a human antibody or that are substantially those of a human antibody in order to reduce the likelihood of eliciting an immune response in the individual that is administered the antibodies to treat AMI. Accordingly, antibodies to BMP-1-3 and BMP-1-4 used to treat AMI are preferably fully human antibodies or humanized antibodies. Less preferably, the antibodies are chimeric antibodies. Less preferably, the antibodies are non-human antibodies that lack any domain or region derived from a human antibody.

For use in treating acute myocardial infarction (AMI) in a human individual according to the invention, a composition comprising an antibody molecule to BMP-1-3 or an antibody molecule to BMP-1-4 or a combination of both antibody molecules is prepared using techniques and ingredients well-known in the art for preparing pharmaceutical compositions for administering a therapeutic antibody to human individuals. A composition comprising an antibody to BMP-1-3 or an antibody to BMP-1-4 or a combination of both antibody molecules may be formulated for administration by any of a variety routes or modes of administration. A composition comprising an antibody to BMP-1-3 or an antibody to BMP-1-4 or combination of both antibody molecules may be formulated for parenteral or non-parenteral administration. Preferably, a composition comprising an antibody to BMP-1-3 or an antibody to BMP-1-4 or a combination of both antibody molecules for use in treating AMI is formulated for parenteral administration, for example, but not limited to, intravenous, subcutaneous, intraperitoneal, or intramuscular administration. More preferably, a composition is formulated for intravenous administration. Such parenteral administration is preferably carried out by injection or infusion of the composition.

Compositions comprising an antibody to BMP-1-3 or an antibody to BMP-1-4 or a combination of both antibody molecules for administration to a human individual may comprise an effective amount of either or both antibody molecules in combination with one or more pharmaceutically acceptable components such as a pharmaceutically acceptable carrier (vehicle, buffer), excipient, or other ingredient. By “pharmaceutically acceptable” is meant that a compound, component, or ingredient of a composition is compatible with the physiology of a human individual and also is not deleterious to the effective activity of the BMP-1-3 antibody or BMP-1-4 antibody component or to a desired property or activity of any other component that may be present in a composition that is to be administered to a human individual. Examples of pharmaceutically acceptable carriers include, but are not limited to, water, saline, phosphate buffered saline, dextrose, glycerol, ethanol and the like, as well as combinations thereof. In many cases, it will be preferable to include isotonic agents, including, but not limited to, sugars; polyalcohols, such as mannitol or sorbitol; sodium chloride; and combinations thereof. Pharmaceutically acceptable carriers may further comprise minor amounts of auxiliary substances such as wetting or emulsifying agents, preservatives, or buffers to enhance the shelf life or effectiveness of the composition. An excipient is generally any compound or combination of compounds that provides a desired feature to a composition. The pH may be adjusted in a composition as necessary, for example, to promote or maintain solubility of component ingredients, to maintain stability of one or more ingredients in the formulation, and/or to deter undesired growth of microorganisms that potentially may be introduced at some point in the procedure.

Compositions comprising a BMP-1-3 antibody or a BMP-1-4 antibody or a combination of both antibody molecules may also include one or more other ingredients such as other medicinal agents (for example, an antibiotic, an anti-inflammatory compound, an anti-viral agent, an anti-cancer agent), fillers, formulation adjuvants, and combinations thereof.

The compositions according to the invention may be in a variety of forms. These include, but are not limited to, liquid, semi-solid, and solid dosage forms, dispersions, suspensions, tablets, pills, powders, liposomes, and suppositories. The preferred form depends on the intended route of administration. Preferred compositions are in the form of injectable or infusible solutions, such as compositions similar to those used administration of therapeutic antibodies approved for use in humans (for example, as used for the therapeutic TNF-α antibody molecules adalimumab or infliximab). In a preferred embodiment, a BMP-1-3 antibody or a BMP-1-4 antibody or a combination of both antibody molecules is administered by intravenous injection or infusion. In another embodiment, an antibody is administered by intramuscular or subcutaneous injection.

Therapeutic compositions must be sterile and stable under the conditions of manufacture and storage. The composition can be formulated as a solution, microemulsion, dispersion, liposome, or other structure suitable for high drug concentration. Sterile injectable solutions may be prepared by incorporating the active compound, i.e., an antibody to BMP-1-3 or antibody to BMP-1-4 or a combination of both antibody molecules, in the required amount in an appropriate solvent, optionally with one or a combination of ingredients that provide a beneficial feature to the composition, as required, followed by filtered sterilization. Generally, dispersions are prepared by incorporating the active ingredient into a sterile vehicle that contains a basic dispersion medium (for example, sterile water, sterile isotonic saline, and the like) and optionally one or more other ingredients that may be required for adequate dispersion. In the case of sterile, lyophilized powders for the preparation of sterile injectable solutions, preferred methods of preparation include vacuum drying and spray-drying that produce a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, a monostearate salt and/or gelatin.

An antibody to BMP-1-3 or an antibody to BMP-1-4 or a combination of both antibody molecules may be administered by a variety of methods known in the art, although a preferred route or mode of administration is parenteral administration and, more preferably, intravenous administration. As will be appreciated by the skilled artisan, the route or mode of administration will vary depending upon the desired results. In certain embodiments, an antibody may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, a polyanhydride, a polyglycolic acid, a collagen, a polyorthoester, and a polylactic acid. A variety of methods for the preparation of such formulations are known to those skilled in the art.

Antibody molecules that bind BMP-1-3 or BMP-1-4 can be employed in any of a variety of antibody-based, immunodetection systems and formats available in the art for detecting a desired antigen in vitro or in vivo. Such systems and formats are readily adapted for detecting or measuring BMP-1-3 or BMP-1-4 in any of a variety of compositions, including, but not limited to, whole blood, plasma, serum, various tissue extracts, and bodily fluids. Examples of such systems or formats that may be adapted for detecting BM-1-3 or BMP-1-4, include, but are not limited to, immunoblots (e.g., Western blots, dot blots), enzyme-linked immunosorbent assays (“ELISAs”), radioimmunoassays (“RIAs”), immunoprecipitations, affinity methods, immunochips, and the like.

According to the invention, there is provided a method for diagnosing acute myocardial infarction (AMI) in a human individual comprising assaying the blood of the individual for the presence of BMP-1-4, wherein detection of BMP-1-4 in the blood indicates that the individual has sustained AMI. In a preferred embodiment, a BMP-1-4 antibody is used to detect BMP-1-4 in the blood of the individual. It may be possible to detect the presence of BMP-1-4 in vivo while circulating in the periphery, e.g., using appropriate imaging systems and a BMP-1-4 antibody that is attached to an appropriate detectable label. However, in a more preferred embodiment of a method for diagnosing AMI in a human individual, a sample of blood is obtained from the human individual and assayed in vitro for the presence of BMP-1-4.

In a typical immunoassay format, a sample of blood obtained from an individual is brought into contact with a BMP-1-4 antibody molecule. The formation of a binding complex between a BMP-1-4 antibody molecule and a BMP-1-4 protein present in the sample of blood is then detected using any of a variety of detection systems available in the art for detecting antibody-antigen binding complexes.

A BMP-1-4 antibody used to detect or to measure the amount of (i.e., quantitate) BMP-1-4 present in blood may be used in solution or alternatively may be immobilized on the surface of any of a variety of solid substrates. Solid substrates to which a BMP-1-4 antibody may be immobilized for use in the methods and compositions described herein include, but are not limited to, magnetic matrix particles; chromatographic matrix or resin particles (e.g., agarose); the surface of one or more wells of a plastic assay plate (such as a microtiter assay plate); pieces of a solid substrate material, such as pieces or strips of plastic, nylon, wood, paper, or other solid material, which may be dipped into or otherwise placed in contact with a blood sample or assay solution; and the surface of a silicon chip (or other chip material). Immobilization of a BMP-1-4 antibody to the surface of the wells of a microtiter plate or the surface of a chip (e.g., a silicon chip, glass slide, etc.) permits the use of formats for detecting or measuring the amount of BMP-1-4 in one or multiple blood samples using semi-automatic or fully automatic devices that are routinely used in standard high throughput ELISA or biochip assay procedures. Such devices are particularly useful for assaying large numbers of very small volumes of blood for the presence of BMP-1-4.

A BMP-1-4 antibody may be immobilized to the surface of a solid substrate by any means that preserves the ability of the antibody to bind to BMP-1-4 when brought into contact with a sample of blood that contains BMP-1-4 to form a binding complex. For example, an antibody may be immobilized to a solid substrate by adsorption (non-covalent adherence) or by covalently linking the antibody directly to the solid surface or to a linker molecule that permits the antibody to be tethered to the solid substrate using methods available in the art.

Methods to detect a binding complex comprising BMP-1-4 and a BMP-1-4 antibody preferably employ a detection system that uses one or more signal-generating molecules (detectable labels) that will generate a signal that is easily detected by the human eye or is readily detected or measured by a signal detection instrument (for example, spectrophotometer). Such signals useful in detecting binding complexes include, but are not limited to, a fluorescent signal, e.g., as generated from a fluorescent dye or cyanin molecule that can be attached directly or indirectly to a BMP-1-4 antibody; a visible color signal, e.g., as generated with an enzyme or colored molecule (e.g., a pigment) that can be attached directly or indirectly to a BMP-1-4 antibody; a radioactive signal, e.g., as generated by a radioisotope that can be attached directly or indirectly to a BMP-1-4 antibody; and a light signal, e.g., as generated by a chemiluminescent or bioluminescent system. An example of a bioluminescent system is a luciferin-luciferase system in which a luciferase may be attached directly or indirectly to an antibody to generate a detectable light signal in the presence of the luciferin substrate.

A detectable label may be conjugated to a BMP-1-4 antibody directly or via a linker molecule using standard reagents and protocols available in the art. Alternatively, a BMP-1-4 antibody may be unlabeled and a secondary binding molecule (for example, an antibody), which binds either the BMP-1-4 antibody or that binds BMP-1-4 in the antigen-antibody binding complex at an epitope not bound by the first BMP-1-4 antibody, may be used to generate a detectable signal. This format is exemplified by the standard sandwich immunoassay in which a “capture antibody” (e.g., BMP-1-4 antibody) binds an antigen of interest (e.g., BMP-1-4) to form a binding complex and a secondary antibody (detection antibody) comprising a detectable label is then provided that binds the capture antibody or binds to the antigen of interest in the binding complex at an epitope that is not bound by the capture antibody. It is understood that if the secondary antibody is also a BMP-1-4 antibody, then it must both bind to an epitope on BMP-1-4 that is not bound by the capture antibody and that is exposed (accessible) on the binding complex formed between the capture antibody and BMP-1-4. Other variations of the sandwich immunoassay are known to the skilled practitioner and adaptable for use in the methods described herein.

In another assay format, BMPM-1-4 in a sample of blood is detected using an assay strip to which a BMP-1-4 antibody is adsorbed or covalently linked. Such assay strips provide a convenient means to detect or measure BMP-1-4 in a sample of blood. For example, an assay strip containing immobilized BMP-1-4 antibody may be brought into contact with a blood sample by manually or robotically dipping the strip into the sample or dropping a sample of blood on the strip. Preferably, the assay strip is first dipped into a blocking agent, such as bovine serum albumin or other composition, to reduce nonspecific binding by potentially interfering molecules. If necessary, the assay strip may be further dipped or contacted with any reagent that is necessary to develop or generate a detectable or measurable signal that indicates the presence on the strip of a binding complex comprising BMP-1-4 bound to the immobilized BMP-1-4 antibody. The assay strip is then observed visually or read by an appropriate detection instrument to determine the presence or amount of BMP-1-4 in the sample.

A method described herein for detecting BMP-1-4 in the blood from an individual may employ whole blood or a fraction of the whole blood, such as plasma or serum. The ultimate determination of whether to use whole blood, plasma, or serum, or even some other blood fraction, in any particular assay format is well within the understanding and judgment of persons of ordinary skill in the art. Generally, plasma is preferred.

The use of standard methods and equipment for obtaining blood samples from individuals, including, without limitation, sterile needles, sterile syringes, sterile partially evacuated blood sample tubes, for obtaining blood samples from human individuals are well known by phlebotomists and healthcare providers.

To accurately measure (quantitate) the level (amount, concentration) of BMP-1-4 in a sample of blood obtained from an individual (and, thereby, in the circulation of the individual), a standard curve may be generated graphically or computationally using an assay as described herein. For example, an assay described herein may be carried out on one or more blood samples and on a series of solutions containing known concentrations of BMP-1-4 or of a peptide or collection of peptides containing one or more epitopes of BMP-1-4 (BMP-1-4 standards). The signal intensity or magnitude obtained for each BMP-1-4 standard is then used to construct a standard curve that correlates the signal intensity or magnitude with an amount or concentration of BMP-1-4. The signal intensity or magnitude from a sample of unknown BMP-1-4 content may then be read on the standard curve to determine the corresponding level (amount, concentration) of BMP-1-4 present in the sample. Preferably, the level of BMP-1-4 in a sample of unknown BMP-1-4 content is determined by interpolation, i.e., by reading a signal magnitude or intensity from the sample of unknown BMP-1-4 content on an area of the standard curve generated or drawn between at least two BMP-1-4 standard points. Less preferred, but optionally, the determination of the amount of BMP-1-4 in a sample may be made by extrapolation, wherein the magnitude or intensity of a signal falls on an area of the standard curve that is drawn or generated beyond or outside of two or more BMP-1-4 standard points.

Methods and compositions described herein preferably employ a BMP-1-4 antibody as the preferred BMP-1-4 binding partner to detect the presence of or quantitate BMP-1-4 in a sample of blood. Nevertheless, it is also understood that such methods and compositions may comprise the use of a BMP-1-4 binding partner other than a BMP-1-4 antibody molecule if that binding partner can be similarly employed or adapted for use in the methods and compositions.

Materials necessary for detection of BMP-1-4 in a sample of blood may be conveniently assembled into a kit that permits a healthcare provider to determine whether an individual has sustained an acute myocardial infarction (AMI). In one embodiment, a kit of the invention comprises a BMP-1-4 antibody and instructions that indicate how to use the kit to carry out an assay to detect BMP-1-4 in a sample of blood. In another embodiment, a kit may comprise a first antibody that binds BMP-1-4 antibody (capture antibody); a second antibody molecule (detection antibody), wherein the second antibody contains a detectable label and binds to the capture antibody or binds to an epitope of BMP-1-4 that is not bound by the capture antibody; and instructions that indicate how to use the kit to carry out the assay to detect or quantitate BMP-1-4 in a sample of blood. The BMP-1-4 antibody used as the capture antibody in a kit may be used in a solution or may be immobilized on a solid substrate, such as a chip, bead, assay strip, surface of the wells of a microtiter plate, and the like, which can be brought into contact with a sample of blood. The component capture antibody and detection antibody in a kit described herein may be packaged in any of a variety of conditions such as a dry state, an unhydrated state, a freeze-dried state, a dehydrated state, or a hydrated state in a physiological buffer solution. Solutions for hydrating, washing, blocking nonspecific binding, or for signal generation from the detectable label on the detection antibody may also be included in a kit described herein. A kit may also include one or more devices to obtain a sample of blood from a human individual. Such a device includes but is not limited to a sterile pin, a sterile needle, a sterile needle and syringe, and a sterile evacuated blood sample tube.

Additional embodiments and features of the invention will be apparent from the following non-limiting examples.

### EXAMPLES

### Example 1

### Identification of BMP-1-3 and BMP-1-4 Isoforms, but not Authentic Osteogenic BMPs, from Human Blood Plasma by Heparin Sepharose Affinity Chromatography, and Protein Identification Using Liquid Chromatography-Mass Spectrometry (“LC-MS”)

The analysis of blood from human subjects for BMP-1-3 and BMP-1-4 isoforms was carried out as previously described. See, Grgurevic et al. (2011); international publication No. WO2008/011193.

### Plasma Collection

Blood samples were collected from healthy adult human volunteers and patients that had sustained an acute myocardial infarction (AMI). The blood samples were drawn into syringes containing 3.8% sodium citrate to form an anticoagulant-to-blood ratio (v/v) of 1:9. Plasma was obtained by centrifugation (15 min. at 3000×g), and aliquots of each adult blood sample were used to make a pooled plasma stock. Aliquot samples were stored at −80° C. prior to analysis.

### Affinity Column Purification

Pooled human plasma (80 ml) was diluted two-fold with 10 mM sodium phosphate buffer (pH 7), and applied to a 5 ml heparin Sepharose column (Amersham Pharmacia Biotech) previously equilibrated with 10 mM sodium phosphate buffer (pH 7). Bound proteins were eluted from the column with 10 mM sodium phosphate buffer (pH 7) containing 1.0 M and 2.0 M NaCl.

### Ammonium Sulfate Precipitation

Saturated ammonium sulfate (“SAS”) was added into the protein eluate drop-by-drop with mixing on a vortex to a final concentration of 35% (w/v). Samples were kept on ice for 10 minutes, and centrifuged for 5 minutes at 12,000×g. The supernatant was discarded, and the pellet was prepared for subsequent analysis by sodium dodecyl sulfate-polyacrylamide gel electrophoresis (SDS-PAGE).

### SDS-PAGE and Western Blot Analysis of the Purified Protein

The pellet was run on standard SDS-polyacrylamide gel electrophoresis (SDS-PAGE) using a 10% gel according to the method of Laemmli (Nature, 227: 680-685 (1970)). After electrophoresis, one part of the SDS-PAGE gel was transferred to nitrocellulose and the other was directly stained with Coomassie Brilliant Blue (“CBB”). Nitrocellulose membrane was first incubated with an antibody specific for an authentic osteogenic BMP, such as BMP-7 (Genera Research Laboratory), or a BMP-1 isoform and kept overnight at 4° C. Alkaline phosphatase-conjugated goat anti-mouse antibody was used as secondary antibody for 1 hour at room temperature. The membrane was developed with 5 ml of a chromogenic substrate. The other part of the gel was stained with Coomassie Brilliant Blue (CBB) under standard staining procedure (0.1% CBB in 45% methanol, 10% acetic acid; 30 minutes at room temperature).

The gel was cut into slices corresponding to each protein band as revealed by staining with CBB. The gel slices were then processed to determine what proteins were present in each slice using a method of analyzing tryptic peptides released from each protein band by HPLC and mass spectrometry (“MS”) using a nanoelectrospray LC-MS interface as described by Olsen and Mann (Proc. Natl. Acad. Sci. USA, 101: 13417-13422 (2004)) as modified by Grgurevic et al. (J. Nephrol., 20: 311-319 (2007)). Aspects of the steps of this method that are specifically related to this study are indicated below.

### In-Gel Trypsin Digestion Protocol

Bands in the gel were excised from CBB stained gels and digested with trypsin. Briefly, gel pieces were shrunk with 100 μl of acetonitrile for 8 minutes. Liquid was removed and gel pieces were re-swelled with 100 μl of ammonium hydrogencarbonate for 12 minutes and then dried in SpeedVac for 10 minutes. Dithiothreitol (“DTT”, 100 μl) was added and incubated for 45 minutes at 57° C. Gel pieces were shrunk with 100 μl of acetonitrile for 8 minutes at 57° C., spun down, and liquid was removed. Iodoacetamide (100 μl) was added to each gel piece and incubated for 45 minutes at room temperature in the dark without agitation. Trypsin (10 μl) was added per gel piece. Then the gel pieces were spun down and re-swelled for 10 minutes. Samples were incubated overnight at 37° C. in a thermo-mixer.

### Peptide Extraction Protocol

Samples were removed from the 37° C. thermo-mixer. A solution (50 μl) containing acetonitrile, water, and formic acid was added. Samples were sonicated for 15 minutes. Supernatant was transferred to the reserve tube, and 50 μl of acetonitrile were added. Extracts were dried under vacuum in the SpeedVac to complete dryness (about 40 minutes). Peptides were re-dissolved with 10 μl of solution containing water, methanol, and formic acid. Samples were sonicated for 5 minutes, and stored at −20° C. until analysis.

### Mass Spectrometry

Tryptic peptides were analyzed by liquid chromatography-mass spectrometry (LC-MS) as follows. Agilent 1100 nanoflow HPLC system (Agilent Technologies, Palo Alto, Calif.) was coupled to a 7-Tesla LTQ-FT mass spectrometer (Thermo Electron, Bremen, Germany) using a nanoelectrospray LC-MS interface (Proxeon Biosystems, Odense, Denmark). Peptides were separated on a home-made 75 μm C₁₈ HPLC column and mass-analyzed on-the-fly in the positive ion mode. Each measurement cycle consisted of a full mass spectrometry (MS) scan, followed by selected ion monitoring (SIM) scan, MS/MS, and MS/MS/MS scans of the three most intense ions. This provided a typical peptide mass accuracy of 2 ppm, as well as additional sequence information from the MS/MS and MS/MS/MS fragment ions. Resulting spectra were centroided, and searched against NCBInr database using Mascot search engine (Matrix Science). Searches were done with tryptic specificity, carboxyamidomethylation as fixed modification, and oxidized methionine as variable modification. Mass tolerance of 5 ppm and 0.6 Da was used for MS and MS/MS spectra, respectively.

### Results

For blood from healthy human individuals, the LS-MS and immunoblotting analyses revealed twelve (12) tryptic peptides that were compared with the NCBInr database. Two peptides were found not to belong to any known authentic osteogenic BMP, but to the splice isoform 3 of the precursor of BMP-1-3 (Swiss-Prot: P13497-2; SEQ ID NO:2), i.e., procollagen C-proteinase. The amino acid sequences of the two peptides are:

(amino acids 653-660 of SEQ ID NO: 2)
S-G-L-T-A-D-S-K, Mascot Score = 36;
(amino acids 280-289 of SEQ ID NO: 2)
G-I-F-L-D-T-I-V-P-K, Mascot Score = 26.

No other protein in the NCBInr database matched the same set of peptides. No authentic osteogenic BMP proteins were detected at molecular weight of 100 kDa and 35 kDa by LS-MS or by immunoblotting. Consistent with previous findings (WO 2008/011193 A2; Grgurevic et al. (2011)), the results indicate that authentic osteogenic BMPs do not normally circulate in the blood of healthy adult humans, whereas BMP-1-3, i.e., a procollagen C-proteinase isoform, is a soluble protein component of normal human blood.

For blood of human patients that had sustained an acute myocardial infarction, the LS-MS and immunoblotting analyses revealed peptides of BMP-1-3 and also two tryptic peptides that were compared with the NCBInr database. The two peptides were found to belong to the amino acid sequence of the BMP-1-4 isoform (SEQ ID NO:3). The amino acid sequences of the two peptides are:

(amino acids 203-216 of SEQ ID NO: 3)
K-N-C-D-K-F-G-I-V-V-H-E-L-G, Mascot Score = 51;
(amino acids 244-256 of SEQ ID NO: 3)
G-V-L-H-S-S-L-L-L-L-S-C-G, Mascot Score = 64.

Thus, whereas the blood of healthy individuals contains BMP-1-3 (and no other BMP-1 isoform), the blood of human individuals that have sustained an acute myocardial infarction contains both BMP-1-3 and BMP-1-4. This is the first time that the BMP-1-4 isoform protein has been demonstrated at the protein level and shown to be present in the blood of human patients of acute myocardial infarction but not in the blood of healthy individuals. Accordingly, the detection of BMP-1-4 in a sample of blood of a human individual indicates that the individual has sustained an acute myocardial infarction.

### Example 2

### Localization of BMP-1-4 in the Human Heart

BMP-1-4 was been localized in the heart and placenta of the developing human embryo using BMP-1-4 antibody (data not shown). In adult human heart sections, BMP-1-4 protein was detected in muscular fibrils and myocytes, but not in tissues from a variety of other major organs (data not shown). The results indicate that expression of the BMP-1-4 isoform is uniquely related to the development and function of the human heart.

### Example 3

### Materials and Methods for Studying Treatments for Acute Myocardial Infarction

### Production of Antibodies

Polyclonal and monoclonal antibodies against BMP-1-3 and BMP-1-4 were generated using synthetic peptide fragments derived from the BMP-1-3 and BMP-1-4 amino acid sequences (SEQ ID NO:2 and SEQ ID NO:3, respectively).

For producing monoclonal antibodies to the BMP-1-3 protein, mice were immunized with a synthetic peptide having the following amino acid sequence of the C-terminal region of the BMP-1-3 protein:

(amino acid residues 972-986 of SEQ ID NO: 2)
R-Y-T-S-T-K-F-Q-D-T-L-H-S-R-K.

For producing monoclonal and polyclonal antibodies to the BMP-1-4 protein, animals were immunized with a synthetic peptide having the following amino acid sequence:

(SEQ ID NO: 8)
C-G-S-R-N-G-A-S-F-P-S-S-L-E-S-S-T-H-Q-A.

This peptide has an amino acid sequence that is identical to amino acid residues 255-274 of BMP-1-4 (SEQ ID NO:3), except that a cysteine (Cys) at position 265 has been replaced by a serine (Ser). This prevented formation of a sulfhydryl cross-link, which may have occluded the desired immunogenic site for generating the anti-BMP-1-4 antibody.

Peptide-specific antibodies were identified using enzyme-linked immunosorbent assay (ELISA) with purified recombinant BMP-1-3 (Genera Research Lab). The antibodies were affinity purified.

Monoclonal antibodies to BMP-1-3 and to BMP-1-4 were obtained from ProMab (Richmond, Calif., USA) using the above peptides to immunize Balb/C mice in the manufacturer's hybridoma procedure. Neutralizing activity of BMP-1-3 antibodies was demonstrated by inhibition of BMP-1-3-mediated cleavage of procollagen or dentin matrix protein-1 (DMP-1) using standard cleavage assays (data not shown). See, for example, Kessler et al. (1996) and Li et al. (1996) (procollagen 1 cleavage assay); Qin et al. (2003) and Steiglitz et al. (2004) (DMP-1 cleavage assay). As shown in the studies described below, both BMP-1-3 mAb and BMP-1-4 mAb were effective in treating acute myocardial infarction in the rat model for the disease.

### Rat Model for Acute Myocardial Infarction

The studies described herein employed the experimental acute myocardial infraction model in rats. This model presents physiopathological alterations that are similar to those that occur after acute myocardial infarctions in humans and is the model of choice for the study of therapeutic interventions to minimize morphological and functional alterations that can occur after the infarction. See, for example, Zornoff et al. (2009). Six-month old Sprague-Dawley rats were initially housed under standard conditions of constant temperature (25° C.) and day-night light cycle. Male rats weighting 250-300 grams were anesthetized with a combination of xylazine (0.6 ml/kg, Rompun®, Bayer AG, Leverkusen, Germany) and ketamine (Narketan, 0.8 ml/kg, Chassot GmbH, Germany) administered intraperitoneally. After a left-side thoracotomy was performed at the fourth intercostal space, the pericardium was incised. The heart was exteriorized through lateral compression of the chest. A ligature (6/0 Ethilon™ suture, Ethicon, Somerville, N.J., USA) was placed around the left main coronary artery close to its origin and between the left atrium border and the pulmonary artery sulcus. Then, the heart was rapidly returned to the thoracic cavity, and the lungs were expanded with positive ventilation.

### Measurement of Plasma CK-MB and Troponin t

Myocardial cellular damage and necrosis were evaluated in rats subjected to ligature-induced acute myocardial infarction (AMI) as described herein by measuring plasma levels of two cardiac markers, i.e., creatine kinase myocardial band (“CK-MB”) and troponin t (“Tn-T”), which are established markers for heart tissue damage. Elevated levels of CK-MB and Tn-T in the blood are indicative of heart tissue damage, including heart tissue damage from acute myocardial infarction. Blood samples were drawn from the orbital plexus of the animals and collected in heparinized tubes. Samples were promptly centrifuged at 2000×g for 15 minutes until measurements were taken. The two markers for AMI were measured using enzyme-linked immunosorbent assays (ELISA).

Levels of toponin t (“Tn-T”) in blood samples were determined in the studies below using a commericial ELISA kit (Troponin T hs STAT, Roche Diagnostics, Mannheim, Germany). This particular ELISA is a quantitative sandwich enzyme immunoassay that employs microtiter plates with wells that have been pre-coated with antibody specific for Tn-T. Standards and samples are added to the wells and any Tn-T present in the standards or samples is bound by the immobilized antibody. After removing any unbound substance, a biotin-conjugated antibody, which is also specific for Tn-T, is added to the wells. After washing, an avidin-conjugated horseradish peroxidase (HRP) is added to the wells. The HRP enzyme substrate TMB (3,3′,5,5′ tetramethyl-benzidine) is then add to the wells to initiate the HRP reaction. During the incubation period, the HRP reaction generates a color in proportion to the amount of Tn-T bound in the initial step to the well of the microtiterplate. The reaction is terminated with a stop solution (sulfuric acid solution), and the color of the reaction mixture in each well is measured spectrophotometrically at 450 nm±2 nm. The concentration of Tn-T in the samples is then determined by comparing the O.D. of the samples to a standard curve.

Levels of CK-MB in blood samples were also determined using an ELISA kit (Creatine Kinase-MB, CKMBL, Roche Diagnostics, Indianapolis, Ind., USA).

### Echocardiographic Assessment

All animals (rats) in the studies below underwent echocardiography under anesthesia. Animals were lightly anesthetized with ketamine and xylazine combination. Two-dimensionally (2D)-guided M-mode transthoracic echocardiography was performed. For M-mode recordings, the parasternal short-axis view was used to image the heart in 2D at the level of the papillary muscle. Left ventricle (LV) volumes were calculated via 2D measurements by a formula. The following M-mode measurements were determined: LV (left ventricular) internal dimensions at both diastole and systole (LVIDd and LVIDs, respectively), LV posterior wall dimensions at diastole and systole (LVPWd and LVPWs, respectively), and interventricular septal dimensions at both diastole and systole (IVSd and IVSs, respectively). From these measurements, ejection fraction (EF) and fractional shortening (FS) were derived. Echocardiography was performed three times on each animal by two different physicians, and the results were presented as mean values.

### PET Data Acquisition and Data Analysis

Positron emission tomography (PET) is a nuclear medicine imaging technique that produces a three-dimensional (3D) image of specific functional processes in the body by following the distribution in space and time of a radiologically marked bioactive molecule injected into the experimental animal. The system detects pairs of gamma rays created by the annihilation of a positron coming from a positron-emitting radionuclide (tracer) attached to the bioactive molecule, and three-dimensional images of tracer concentration within the body are then reconstructed by computer analysis. In the studies of acute myocardial infarction described herein, the bioactive molecule was fludeoxyglocose (FDG, fluorodeoxyglucose), which is an analogue of glucose that could be intravenously injected into the experimental animals. FDG is taken up by functional myocardial tissue, but not by non-functional ischemic myocardial tissue. The technique depends on coincident detection of the pair of photons moving in approximately opposite directions (it would be exactly opposite in their center of mass frame).

The studies described herein were concerned with processes that occurred over a relatively long time period. In particular, for each animal in an acute myocardial infarction study, measurements were taken to obtain images at three time points: (1) prior to experiment to establish the base line for each animal, (2) after the ligation which produced the ischemic effect (1 week), and much longer after the operation (1 month). In this way, all stages of a recovery process were covered: base line (normal uptake), acute stage (immediately after operation), and long-term recovery.

Data analysis was performed using 3.3 version of PMOD software, which was synchronized and calibrated with respect to the input from a ClearPet camera.

The analysis was divided into two steps: (1) qualitative analysis and (2) quantitative analysis. To establish the qualitative effect, the following procedure was devised: First, the time average of acceptable time frames was determined. Then a FUSION PMOD program was used to co-register different measurements (bring them into identical position). For the acute myocardial infarction experiments, all measurements were co-registered with the base line measurements. Next, a 3D PMOD program in a SURFACE type mode to obtain a 3D image of the heart for each experiment, but varied the surface threshold from the smallest value to higher ones to depict the isoactivity lines in all animals.

### Example 4

### Analysis of CK-MB Enzyme in Plasma of Rats with Acute Myocardial Infarction Treated with a Monoclonal Antibody to BMP-1-3

This study determined plasma levels of creatine kinase myocardial band (CK-MB) protein in rats with ligation-induced acute myocardial infarction treated with monoclonal antibody to BMP-1-3 (BMP-1-3 mAb) before and after surgery. A total of 16 rats were used. The animals were divided into a control group, which consisted of 6 animals, and a therapy group consisting of 10 rats that were pretreated with BMP-1-3 monoclonal antibody (15 μg/kg). After surgery, the surviving animals were divided into two groups: (1) control rats with ligated coronary artery (n=4) and (2) rats with ligated coronary artery and treated with BMP-1-3 monoclonal antibody every day during the first week (n=7). Blood was collected at different time points: prior to surgery, and first, second, third, and seventh day after surgery. As shown in FIG. 2, CK-MB values before the ligation were similar, while 24 hours (1 day) after surgery the values were lower in the antibody-treated rats (376.7 U/L in group treated with BMP-1-3 mAb versus 459 U/L in untreated control group). On the second day, the CK-MB was 621.8 in untreated control rats while it was only 441.9 in rats treated with BMP-1-3 mAb (p<0.05). At later time points, CK-MB values also were lower in rats treated with BMP-1-3 mAb compared to untreated control rats. See, FIG. 2.

The results indicate that BMP-1-3 is a therapeutic target for acute myocardial infarction and that administration of an antibody to BMP-1-3 is an effective therapy for treating an individual for acute myocardial infarction.

### Example 5

### Analysis of CK-MB Enzyme in Plasma of Rats with Acute Myocardial Infarction Treated with a Polyclonal Antibody to BMP-1-4

A total of 20 rats were used in this experiment. The level of CK-MB in the blood of rats at different time points was measured: before coronary artery ligation surgery and first, second, third, sixth, and seventh day post-ligation). Ten rats were pretreated with BMP-1-4 polyclonal antibody (15 μg/kg). After surgery, the rats that survived the coronary ligation were divided into two groups: (1) control rats with ligation-induced acute myocardial infarction without therapy (n=8) and (2) rats with ligation-induced acute myocardial infarction and treated with BMP-1-4 polyclonal antibody (n=6). As shown in FIG. 3, treatment with a BMP-1-4 polyclonal antibody significantly decreased the serum value of CK-MB in rats 2 days after surgery compared to untreated control rats (e.g., 563.8 in control group versus 441.5 in antibody-treated rats). At later time points, CK-MB values also were lower in rats treated with the BMP-1-4 polyclonal antibody compared to untreated control rats (e.g., 395.5 in control group compared to 237.2 in rats treated with the BMP-1-4 polyclonal antibody on day 7).

The results indicate that BMP-1-4 is a therapeutic target for acute myocardial infarction and that administration of an antibody to BMP-1-4 is an effective therapy for treating acute myocardial infarction.

### Example 6

### Analysis of Troponin t in Plasma of Rats with Acute Myocardial Infarction Treated with Combination of BMP-1-3 and BMP-1-4 Monoclonal Antibodies

This study followed the level of troponin t (“Tn-T”) in rats with induced acute myocardial infarction (AMI) that were treated with a combination of a BMP-1-3 mAb and a BMP-1-4 mAb, before and after the ligation surgery to induce AMI. A total of 21 rats were used in this study. Prior to coronary artery ligation, seven rats were pretreated with a combination (BMP-1-3 antibody+BMP-1-4 antibody; 15 μg/kg for each antibody), while fourteen rats remained untreated (control group). After 24 hours rats, underwent surgical ligation of the left coronary artery. The surviving animals were divided into two groups: (1) control rats with ligated coronary artery (n=6) and (2) BMP-1-3 mAb+BMP-1-4 mAb treated rats with induced AMI (n=3). Antibody-treated animals received 15 μg/kg of each antibody at 24 and 48 hours after surgery. Blood was collected at different time points: prior to surgery, first day, second day, third day, and sixth day after the ligation surgery (FIG. 4). The combination of antibodies showed a significant efficacy in decreasing serum Tn-T levels relative to untreated control animals with AMI. During the first, second, and third days, the values in untreated control rats were 7.8, 3.26, 1.18, whereas in antibody-treated animals, the values were 5.72, 1.63, and 0.24. See, FIG. 4.

The results indicate that a combination therapy of BMP-1-3 mAb and BMP-1-4 mAb is effective for treating acute myocardial infarction.

### Example 7

### Echocardiographic Assessment of Heart Function in Rats with Acute Myocardial Infarction

The functional consequence of antibody therapy and formation of fibrosis was further studied by cardiac echocardiography in the M mode of untreated coronary-ligated control rats and of coronary-ligated rats treated with a BMP-1-3 monoclonal antibody (15 μg/kg). A total of 19 rats were used for this long-term follow-up study. After surgery survived rats were divided into three groups: (1) sham operation group: normal, healthy animals (n=3), (2) control group: untreated rats with induced acute myocardial infarction (n=4), (3) therapy group: rats with induced acute myocardial infarction treated with BMP-1-3 monoclonal antibody before and during the first week after the surgery (n=7). Echocardiography was performed 45 days after surgery. Analyses of healthy rats were used according to define the mean values as follows: IVSd=1.1 mm, LVIDd=5.3 mm, LVPWd=1.77 mm, IVSs=2.3 mm, LVIDs=2.7 mm, LVPWs=2.4 mm, EF=85.5%, FS=49.1%. Control rats with induced acute myocardial infarction had a profound decrease in function, which occurred in echocardiographic parameters: IVSd=1 mm, LVPWd=1.73 mm, IVSs=1.1 mm, EF=68.8%, FS=33.9%. Treatment (therapy) with a BMP-1-3 monoclonal antibody enhanced cardiac function: IVSd=1.38 mm, LVIDd=5.9 mm, IVSs=2.7 mm, EF=88.2%, FS=52.9%. See, Table 1, below.

TABLE 1
Echocardiography measurements of heart dimensions and function
of rats in animal model of acute myocardial infarction.

| Heart | | | BMP1-3 mAb |
|---|---|---|---|
| Parameter | Sham | Control | treatment |
| IVSd (mm) | 1.1 | 1 | 1.4* |
| LVIDd (mm) | 5.3 | 5.5 | 5.9 |
| LVPWd (mm) | 1.7 | 1.7 | 1.7 |
| IVSs (mm) | 2.3 | 1.1 | 2.7* |
| LVIDs (mm) | 2.7* | 3.6 | 2.8* |
| LVPWs (mm) | 2.4 | 5.2 | 2.8* |
| EF (%) | 85.5* | 68.8 | 88.2* |
| FS (%) | 49.1* | 33.9 | 52.9* |

Sham = sham operation rats;
Control = untreated rats subjected to ligation-induced acute myocardial infarction;
BMP1-3 mAb = BMP-1-3 monoclonal antibody treatment of rats subjected to ligation-induced acute myocardial infarction;
*p < 0.05 (statistical significance as compared to control rats)

### Example 8

### Positron Emission Tomography (PET) Data Acquisition and Data Analysis

In this experiment, 20 rats were scanned by PET prior to surgery in the rat model of acute myocardial infarction. Before the surgery, the rats were divided into control rats (n=10) and animals treated with a BMP-1-3 mAb (n=10). After surgery, the mortality was 50% in control rats and 30% in rats treated with a BMP-1-3 mAb prior to surgery. Rats were then treated with BMP-1-3 mAb on days 2, 7, and 14 at a dose of 15 μg/kg. Besides the first PET scan prior to the surgery, the rats were scanned after the first week and first month to evaluate the progression of infarction and influence of the therapy. The images of representative hearts of animals from control group and from BMP-1-3 mAb treatment group are shown in FIG. 5. After the first week both groups showed a decreased FDG uptake in the infarcted area (0.36 vs 0.38). After one month in rats treated with a BMP-1-3 mAb, FDG uptake in the infarcted area was restored (0.42) indicative of substantial remodeling and regeneration of functional myocardial tissue in the former infarcted region while in untreated control rats uptake remained low (0.36) indicative of non-functional scar tissue. See, FIG. 5.

### Example 9

### Histological Analysis of the Heart Muscle after Acute Myocardial Infarction

A histological analysis was performed on the heart muscle of rats with ligation-induced acute myocardial infarction (AMI) to assess the effect of treatment with BMP-1-3 monoclonal antibody (BMP-1-3 mAb). Rats (n=16) were divided into a control group, which consisted of six animals, and a therapy group, which consisted of 10 animals that were pretreated with BMP-1-3 mAb (15 μg/kg). After surgery, the surviving animals (n=11) were divided into two groups: (1) control rats with ligated coronary artery (n=4, no pretreatment with BMP-1-3 mAb) and (2) rats with ligated coronary artery that had been pretreated with BMP-1-3 and that were then treated with BMP-1-3 mAb every day during the first week (n=7).

Myocardial tissue from the left ventricle of AMI rats (approximately 2 mm in thickness) was removed. Samples were fixed in 4% pre-cooled paraformaldehyde for 72 hours and embedded in paraffin for histological studies. Paraffin-embedded tissues were sectioned into slices approximately 5 μm thick. Sections were stained with standard hematoxylin and eosin (“H&E”) to reveal cellular components and with Sirius red (and picric acid) to identify fibrous collagen tissue accumulation. Images were visualized under an optical microscope.

FIG. 6 shows micrographs from the histological analysis of the heart muscle of rats following coronary artery ligation for untreated control rats with AMI and rats with AMI that were treated with BMP-1-3 mAb. FIG. 6A shows heart section from the infarcted area of the heart of an untreated control rat at one week after ligation of left coronary artery to induce AMI (4× magnification in FIG. 6A). FIG. 6B shows Sirius red staining of tissue of rectangle area in FIG. 6A (at 20× magnification) indicating early collagen deposition. See, arrows in FIG. 6B. FIG. 6C shows section of myocardial tissue from an untreated rat control with AMI stained with hematoxylin and eosin revealing residual fibrotic scar tissue after 1 month surrounded by damaged myocardial fibers. See arrow in FIG. 6C. FIG. 6D shows a heart section from infarcted area of heart of rat treated with BMP-1-3 mAb (15 μg/kg) prior to ligation of left coronary artery to induce AMI and then treated with BMP-1-3 mAb every day during the first week after surgery. The fibrotic area following AMI was significantly smaller than that observed in control rats. See, arrow in FIG. 6D. FIG. 6E shows a higher magnification of area indicated by arrow in FIG. 6D revealing spots of new regenerative muscle fibers. See, arrows in FIG. 6E. FIG. 6F shows a higher magnification of the area indicated by arrows in FIG. 6E revealing newly formed muscle fibers and surrounding cells with fibrous tissue that was clearly less dense than that observed in tissue from untreated control rats.

The histological analysis of the heart muscle tissue after AMI indicates that treatment with the BMP-1-3 mAb significantly decreased the size of the scar and promoted formation of nodules with newly formed muscle fibers in the original infarct region.

Taken together, the results of the above examples clearly indicate that administration of an antibody to BMP-1-3 and/or an antibody to BMP-1-4 is effective for reducing progression of the original infarct region in the heart of an individual who has sustained an acute myocardial infarction and for promoting remodeling of tissue in the original infarct region to form repair and scar tissue that are of significantly higher quality and more functional than that in the absence of antibody treatment.

All patents, applications, and publications cited in the above text are incorporated herein by reference.

Other variations and embodiments of the invention described herein will now be apparent to those of skill in the art without departing from the disclosure of the invention or the claims below.

1. A method of treating acute myocardial infarction in an individual comprising administering to the individual an antibody to BMP-1-3, an antibody to BMP-1-4, or a combination of an antibody to BMP-1-3 and an antibody to BMP-1-4.

2. The method according to claim 1, comprising administering to the individual an antibody to BMP-1-3.

3. The method according to claim 1, comprising administering to the individual an antibody to BMP-1-4.

4-7. (canceled)

8. A method of determining whether an individual has sustained an acute myocardial infarction comprising assaying a blood sample previously obtained from the individual for the presence of BMP-1-4, wherein the presence of BMP-1-4 in the blood sample indicates that the individual has sustained an acute myocardial infarction.

9. The method according to claim 8, wherein the step of assaying the blood sample for the presence of BMP-1-4 comprises contacting the blood sample with a BMP-1-4 binding partner to form a binding complex between the binding partner and BMP-1-4 present in the blood sample.

10. The method according to claim 9, wherein the binding complex formed between the binding partner and BMP-1-4 present in the blood sample is detected by a detectable label associated with the BMP-1-4 binding partner.

11. The method according to claim 9, wherein the binding complex formed between the BMP-1-4 binding partner and BMP-1-4 present in the blood sample is detected by adding an antibody that binds to said BMP-1-4 binding partner or that binds to said BMP-1-4 present in said binding complex and detecting said antibody by a detectable label present on said antibody.

12. The method according to any one of claims 8-11, wherein the BMP-1-4 binding partner is an antibody that binds BMP-1-4.

13. (canceled)

14. An anti-BMP-1-3 antibody raised against the peptide immunogen R-Y-T-S-T-K-F-Q-D-T-L-H-S-R-K (amino acid residues 972-986 of SEQ ID NO:2).

15. An anti-BMP-1-4 antibody raised against the peptide immunogen C-G-S-R-N-G-A-S-F-P-S-S-L-E-S-S-T-H-Q-A (SEQ ID NO:8).

16. An anti-BMP-1-3 antibody produced by a hybridoma cell line that has accession no. DSM ACC3198.

17. An anti-BMP-1-4 antibody produced by a hybridoma cell line that has accession no. DSM ACC3213.

18. A method of treating an individual to prevent or inhibit damage to myocardial tissue from an acute myocardial infarction comprising administering to the individual an antibody to BMP-1-3, or an antibody to BMP-1-4, or a combination of an antibody to BMP-1-3 and an antibody to BMP-1-4 prior to an acute myocardial infarction.

19. The method according to claim 18, comprising administering to the individual an antibody to BMP-1-3.

20. The method according to claim 18, comprising administering to the individual an antibody to BMP-1-4.


SEQUENCE LISTING (8 sequences)

SEQ ID NO: 1 - protein, 730 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSEKRPALQPPRGRPHQLKFRVQKRNRTPQ

SEQ ID NO: 2 - protein, 986 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSDKDECSKDNGGCQQDCVNTFGSYECQCRSGFVLHDNKHDCKEAGCDHKVTSTSGTITSPNWPDKYPSKKECTWAISSTPGHRVKLTFMEMDIESQPECAYDHLEVFDGRDAKAPVLGRFCGSKKPEPVLATGSRMFLRFYSDNSVQRKGFQASHATECGGQVRADVKTKDLYSHAQFGDNNYPGGVDCEWVIVAEEGYGVELVFQTFEVEEETDCGYDYMELFDGYDSTAPRLGRYCGSGPPEEVYSAGDSVLVKFHSDDTITKKGFHLRYTSTKFQDTLHSRK

SEQ ID NO: 3 - protein, 302 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGVLHSSLLLLSCGSRNGASFPCSLESSTHQALCWTGLFLRPSPFPRLPLAAPRTLRAGV

SEQ ID NO: 4 - protein, 4 aa, Homo sapiens
  MISC_FEATURE (3)..(3): X is any amino acid
FGXG

SEQ ID NO: 5 - protein, 9 aa, Homo sapiens
  MISC_FEATURE (2)..(9): X is any amino acid
CXXXXXXXX

SEQ ID NO: 6 - protein, 5 aa, Homo sapiens
LEWIG

SEQ ID NO: 7 - protein, 4 aa, Homo sapiens
  MISC_FEATURE (3)..(3): X is any amino acid
WGXG

SEQ ID NO: 8 - protein, 20 aa, Artificial Sequence
  modified peptide segment from BMP-1-4
CGSRNGASFPSSLESSTHQA